SEQUENCE LISTING (4 sequences)

SEQ ID NO: 1 - DNA, 2529 bp, Artificial Sequence
  nucleotide sequence encoding the CD19 CAR
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 2 - DNA, 9382 bp, Artificial Sequence
  nucleotide sequence representing the expression vector encoding the CD19 CAR
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 3 - DNA, 3180 bp, Artificial Sequence
  nucleotide sequence encoding the CD20 CAR
atgttgctgctggttacatctctgctgctgtgcgagctgccccatcctgcctttctgctgatccccgacatcgtgctgacacagagccctgccatcctgagtgcttccccaggcgagaaagtgaccatgacctgtagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaagggctctacaagcggcggaggatctggcggtggaagtggcggaggcggatcttctgaagttcagctgcaacagtctggcgccgagctggttaagcctggcgcctctgtgaagatgagctgcaaggccagcggctacaccttcaccagctacaacatgcactgggtcaagcagacccctggacagggactcgagtggatcggagccatctatcccggcaatggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagtgtctagcgagtctaagtacggaccgccttgtcctccttgtccagctcctcctgtggccggacctagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttccagagcacctaccgggtggtgtccgtgctgacagtgctgcaccaggactggctgaacggcaaagagtacaagtgcaaggtgtccaacaagggcctgcccagcagcatcgagaaaaccatcagcaaggccaagggccagcctcgcgagccccaggtgtacacactgcctccaagccaggaagagatgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaagatgttctgggtgctggtggtcgtgggcggagtgctggcctgttacagcctgctcgtgaccgtggccttcatcatcttttgggtcaagcggggcagaaagaagctgctgtatatcttcaagcagcccttcatgcggcccgtgcagaccacacaggaagaggacggctgctcctgccggttccccgaggaagaagaaggcggctgcgagctgagagtgaagttcagcagaagcgccgacgcccctgcctatcagcagggccagaaccagctgtacaacgagctgaacctgggcagacgggaagagtacgacgtgctggataagcggagaggccgggaccctgagatgggcggcaagcctagaagaaagaacccccaggaaggcctgtataacgaactgcagaaagacaagatggccgaggcctacagcgagatcggaatgaagggcgagcggagaagaggcaagggccacgatggcctgtaccagggactgagcaccgccaccaaggatacctatgacgcactgcacatgcaggccctgccccccagactcgagggcggaggcgaaggcagaggatctctgctgacatgcggcgacgtggaagagaaccctggccccagaatgctgctgctcgtgacaagcctgctgctgtgcgagctgccccaccctgcctttctgctgatcccccggaaagtgtgcaacggcatcggcatcggagagttcaaggacagcctgtccatcaacgccaccaacatcaagcacttcaagaattgcaccagcatcagcggcgacctgcacatcctgccagtggcctttagaggcgacagcttcacccacacccccccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 4 - DNA, 10033 bp, Artificial Sequence
  nucleotide sequence representing the expression vector encoding the CD20 CAR
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgttgctgctggttacatctctgctgctgtgcgagctgccccatcctgcctttctgctgatccccgacatcgtgctgacacagagccctgccatcctgagtgcttccccaggcgagaaagtgaccatgacctgtagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaagggctctacaagcggcggaggatctggcggtggaagtggcggaggcggatcttctgaagttcagctgcaacagtctggcgccgagctggttaagcctggcgcctctgtgaagatgagctgcaaggccagcggctacaccttcaccagctacaacatgcactgggtcaagcagacccctggacagggactcgagtggatcggagccatctatcccggcaatggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagtgtctagcgagtctaagtacggaccgccttgtcctccttgtccagctcctcctgtggccggacctagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttccagagcacctaccgggtggtgtccgtgctgacagtgctgcaccaggactggctgaacggcaaagagtacaagtgcaaggtgtccaacaagggcctgcccagcagcatcgagaaaaccatcagcaaggccaagggccagcctcgcgagccccaggtgtacacactgcctccaagccaggaagagatgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaagatgttctgggtgctggtggtcgtgggcggagtgctggcctgttacagcctgctcgtgaccgtggccttcatcatcttttgggtcaagcggggcagaaagaagctgctgtatatcttcaagcagcccttcatgcggcccgtgcagaccacacaggaagaggacggctgctcctgccggttccccgaggaagaagaaggcggctgcgagctgagagtgaagttcagcagaagcgccgacgcccctgcctatcagcagggccagaaccagctgtacaacgagctgaacctgggcagacgggaagagtacgacgtgctggataagcggagaggccgggaccctgagatgggcggcaagcctagaagaaagaacccccaggaaggcctgtataacgaactgcagaaagacaagatggccgaggcctacagcgagatcggaatgaagggcgagcggagaagaggcaagggccacgatggcctgtaccagggactgagcaccgccaccaaggatacctatgacgcactgcacatgcaggccctgccccccagactcgagggcggaggcgaaggcagaggatctctgctgacatgcggcgacgtggaagagaaccctggccccagaatgctgctgctcgtgacaagcctgctgctgtgcgagctgccccaccctgcctttctgctgatcccccggaaagtgtgcaacggcatcggcatcggagagttcaaggacagcctgtccatcaacgccaccaacatcaagcacttcaagaattgcaccagcatcagcggcgacctgcacatcctgccagtggcctttagaggcgacagcttcacccacacccccccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

(19) United States
(12) Patent Application Publication
(10) Pub. No.: US 2020/0209240 A1
(43) Pub. Date: Jul. 2, 2020

(54) CD19CART CELLS ELIMINATE MYELOMA CELLS THAT EXPRESS VERY LOW LEVELS OF CD19

(71) Applicant: JULIUS-MAXIMILIANS-UNIVERSITÄT WÜRZBURG, Würzburg (DE)

(72) Inventors: Hermann EINSELE, Würzburg (DE); Michael HUDECEK, Höchberg (DE); Sebastian LETSCHERT, München (DE); Thomas NERRETER, Würzburg (DE); Markus SAUER, Heidelberg (DE)

(21) Appl. No.: 16/646,986

(22) PCT Filed: Nov. 20, 2018
(86) PCT No.: PCT/EP2018/081924

§ 371 Date: Mar. 12, 2020

(30) Foreign Application Priority Data
Nov. 20, 2017 (EP) 17202583.5

(51) Int. Cl. G01N 33/574; C12N 5/0783; C07K 14/705

(52) U.S. Cl. CPC G01N 33/574; C07K 14/70596; C12N 5/0636

(57) ABSTRACT

The invention generally relates to immunotherapy with chimeric antigen receptor (CAR}- engineered T-cells. In particular, the invention relates immunotherapy with chimeric antigen receptor (CAR)-engineered T-cells to target sub-populations of cancer cells that are characterized by low expression of a cancer cell surface antigen, more particular the invention relates to immunotherapy with chimeric antigen receptor (CAR)-engineered T-cells targeting CD19 (CD19CART) in multiple myeloma, a clonal proliferation of plasma cells.

### FIELD OF THE INVENTION

The invention generally relates to immunotherapy with chimeric antigen receptor (CAR)-engineered T-cells. In particular, the invention relates immunotherapy with chimeric antigen receptor (CAR)-engineered T-cells to target sub-populations of cancer cells that are characterized by low expression of a cancer cell surface antigen, more particular the invention relates to immunotherapy with chimeric antigen receptor (CAR)-engineered T-cells targeting CD19 (CD19CART) in multiple myeloma, a clonal proliferation of plasma cells.

### BACKGROUND OF THE INVENTION

Multiple myeloma (MM) is a hematologic malignancy with clonal proliferation of plasma cells that produce aberrant immunoglobulin. Despite aggressive treatment with polychemotherapy, myeloma remains incurable in the majority of patients¹. In a recent clinical study, Garfall et al reported the clinical efficacy of adoptive immunotherapy with gene-engineered T-cells expressing a chimeric antigen receptor (CAR) specific for the B-cell marker CD19 (CD19CART) in heavily pre-treated myeloma patients. They observed one complete and several partial responses in patients that were treated with CD19CART after myeloablative chemotherapy and autologous hematopoietic stem cell transplantation (HSCT)². Notably, previous myeloablative chemotherapy and autologous HSCT had only induced a partial, transient response in the patient who achieved the complete response, and therefore, this outcome was attributed to the administration of CD19CART².

CD19CART therapy is approved as a potentially curative treatment for patients with relapsed/refractory B-cell acute lymphoblastic leukemia (ALL) and non-Hodgkin's lymphoma (NHL)³⁻⁶. In these diseases, CD19 is uniformly expressed on malignant cells, with an antigen density in the order of several thousands of molecules per cell^{3,4,7}, which is thought to be an optimal range for recognition by CD19CART. In contrast, CD19 is generally considered an infrequently expressed, non-uniform target on myeloma cells^{2,8}. According to conventional detection by flow cytometry (FC), CD19 was only present on 0.05% of myeloma cells in the patient that achieved the complete response in the Garfall et al study, which has sparked controversy over the role of CD19 as a therapeutic target in myeloma. In addition, there is an ongoing debate about the sensitivity of FC and the threshold of CD19 antigen density required for CD19CART activation.

In previous work, the inventors have demonstrated the capacity of direct stochastic optical reconstruction microscopy (dSTORM) to determine absolute copy numbers of molecules on plasma membranes of human cells^{9,10}. This super-resolution microscopy method has single-molecule sensitivityl^{11,12}, suggesting that this technique could be used to detect very low expression levels of CD19 on myeloma cells that would be otherwise undetectable by FC. The inventors hypothesized that CD19 may be expressed on a proportion of myeloma cells at a molecular density below the detection limit of FC. To test this, the inventors used dSTORM to generate expression profiles of CD19 on myeloma cells and assessed their recognition by CD19CART. The inventors show that in a subset of myeloma patients, CD19 is expressed on a large fraction of myeloma cells at a very low antigen density that is below the detection limit of FC and demonstrate that less than 100 CD19 molecules per myeloma cell are sufficient for recognition and elimination by CD19CART.

### DESCRIPTION OF THE INVENTION

The invention generally relates to immunotherapy using immune cells such as chimeric antigen receptor (CAR)-engineered T-cells. In particular, the invention relates to immunotherapy using chimeric antigen receptor (CAR)-engineered T-cells to target sub-populations of cancer cells that are characterized by low expression of a cancer cell surface antigen, more particularly the invention relates to immunotherapy with chimeric antigen receptor (CAR)-engineered T-cells targeting CD19 (CD19CART) in multiple myeloma, a clonal proliferation of plasma cells.

The present invention is exemplified by the following preferred embodiments:

1. A method, comprising steps of:
(A) Analyzing a cancer cell-containing sample from a cancer patient to obtain information about a cell surface antigen of the cancer cell; and

(B) Classifying said cancer cell-containing sample based on the information obtained in step (A).

2. The method of item 1, wherein said cancer is a hematologic or solid tumor.

3. The method of items 1 or 2, wherein said cancer is leukemia, lymphoma, or myeloma, preferably wherein said cancer is multiple myeloma.

4. The method of any one of items 1 to 3, wherein step (A) comprises analyzing the cancer cell-containing sample using super-resolution microscopy.

5. The method of any one of items 1 to 4, wherein step (A) comprises determining the number of molecules of said cell surface antigen on said cancer cell.

6. The method of item 4 or 5, wherein said super-resolution microscopy is single-molecule localization microscopy.

7. The method of items 4 to 6, wherein said super-resolution microscopy is dSTORM, STORM, PALM, or FPALM.

8. The method of item 7, wherein said super-resolution microscopy is dSTORM.

9. The method of any one of items 1 to 8, wherein in step (A) of the method, said cancer cell-containing sample is analyzed as to whether it comprises a fraction of cancer cells which express a cell surface antigen.

10. The method of any one of items 6 to 9, wherein the cell surface antigen is the antigen according to item 5.

11. The method of any one of items 5 to 10, wherein the cell surface antigen is a cancer antigen.

12. The method of any one of items 5 to 11, wherein said cell surface antigen is not detectable by flow cytometry.

13. The method of any one of items 5 to 12, wherein said cell surface antigen is detectable by super-resolution microscopy.

14. The method of any one of items 5 to 13, wherein said cell surface antigen is detectable by single-molecule localization microscopy.

15. The method of any one of items 5 to 14, wherein said cell surface antigen is detectable by dSTORM, STORM, PALM, or FPALM.

16. The method of item 15, wherein said cell surface antigen is detectable by dSTORM.

17. The method of any one of items 5 to 16, wherein in step (A) of the method, said cancer cell-containing sample is analyzed as to whether it comprises a fraction of cancer cells which express said cell surface antigen at a number of more than 4 cell surface antigen molecules per cell.

18. The method of any one of items 5 to 16, wherein in step (A) of the method, said cancer cell-containing sample is analyzed as to whether it comprises a fraction of cancer cells which express said cell surface antigen at a number of more than 8 cell surface antigen molecules per cell.

19. The method of any one of items 5 to 16, wherein in step (A) of the method, said cancer cell-containing sample is analyzed as to whether it comprises a fraction of cancer cells which express said cell surface antigen at a number of more than 16 cell surface antigen molecules per cell.

20. The method of any one of items 5 to 16, wherein in step (A) of the method, said cancer cell-containing sample is analyzed as to whether it comprises a fraction of cancer cells which express said cell surface antigen at a number of more than 32 cell surface antigen molecules per cell.

21. The method of any one of items 5 to 16, wherein in step (A) of the method, said cancer cell-containing sample is analyzed as to whether it comprises a fraction of cancer cells which express said cell surface antigen at a number of more than 64 cell surface antigen molecules per cell.

22. The method of any one of items 5 to 16, wherein in step (A) of the method, said cancer cell-containing sample is analyzed as to whether it comprises a fraction of cancer cells which express said cell surface antigen at a number of more than 100 cell surface antigen molecules per cell.

23. The method of any one of items 5 to 16, wherein in step (A) of the method, said cancer cell-containing sample is analyzed as to whether it comprises a fraction of cancer cells which express said cell surface antigen at a number of more than 200 cell surface antigen molecules per cell.

24. The method of any one of items 5 to 16, wherein in step (A) of the method, said cancer cell-containing sample is analyzed as to whether it comprises a fraction of cancer cells which express said cell surface antigen at a number of more than 300 cell surface antigen molecules per cell.

25. The method of any one of items 5 to 24, wherein in step (A) of the method, said cancer cell-containing sample is analyzed as to whether it comprises a fraction of cancer cells which express said cell surface antigen at a number of no more than 10,000 cell surface antigen molecules per cell.

26. The method of any one of items 5 to 24, wherein in step (A) of the method, said cancer cell-containing sample is analyzed as to whether it comprises a fraction of cancer cells which express said cell surface antigen at a number of no more than 5,000 cell surface antigen molecules per cell.

27. The method of any one of items 5 to 24, wherein in step (A) of the method, said cancer cell-containing sample is analyzed as to whether it comprises a fraction of cancer cells which express said cell surface antigen at a number of no more than 2,500 cell surface antigen molecules per cell.

28. The method of any one of items 5 to 24, wherein in step (A) of the method, said cancer cell-containing sample is analyzed as to whether it comprises a fraction of cancer cells which express said cell surface antigen at a number of no more than 1,500 cell surface antigen molecules per cell.

29. The method of any one of items 5 to 24, wherein in step (A) of the method, said cancer cell-containing sample is analyzed as to whether it comprises a fraction of cancer cells which express said cell surface antigen at a number of no more than 1,350 cell surface antigen molecules per cell.

30. The method of any one of items 5 to 24, wherein in step (A) of the method, said cancer cell-containing sample is analyzed as to whether it comprises a fraction of cancer cells which express said cell surface antigen at a number of no more than 1,300 cell surface antigen molecules per cell.

31. The method of any one of items 5 to 24, wherein in step (A) of the method, said cancer cell-containing sample is analyzed as to whether it comprises a fraction of cancer cells which express said cell surface antigen at a number of no more than 1,000 cell surface antigen molecules per cell.

32. The method of any one of items 5 to 24, wherein in step (A) of the method, said cancer cell-containing sample is analyzed as to whether it comprises a fraction of cancer cells which express said cell surface antigen at a number of no more than 800 cell surface antigen molecules per cell.

33. The method of any one of items 5 to 24, wherein in step (A) of the method, said cancer cell-containing sample is analyzed as to whether it comprises a fraction of cancer cells which express said cell surface antigen at a number of no more than 500 cell surface antigen molecules per cell.

34. The method of any one of items 5 to 24, wherein in step (A) of the method, said cancer cell-containing sample is analyzed as to whether it comprises a fraction of cancer cells which express said cell surface antigen at a number of no more than 400 cell surface antigen molecules per cell.

35. The method of any one of items 5 to 34, wherein said number of molecules of said cell surface antigen per cell is determined by microscopy.

36. The method of any one of items 5 to 35, wherein said number of molecules of said cell surface antigen per cell is determined by super-resolution microscopy.

37. The method of any one of items 5 to 36, wherein said number of molecules of said cell surface antigen per cell is determined by single-molecule localization microscopy.

38. The method of any one of items 35 to 37, wherein said microscopy is dSTORM, STORM, PALM, or FPALM.

39. The method of any one of items 35 to 38, wherein said microscopy is dSTORM.

40. The method of any one of items 1 to 39, wherein said cell surface antigen is selected from the group consisting of CD19, CD20, CD22, CD27, CD30, CD33, CD38, CD44v6, CD52, CD64, CD70, CD72, CD123, CD135, CD138, CD220, CD269, CD319, ROR1, ROR2, SLAMF7, BCMA, αvβ3-Integrin, α4β1-Integrin, EpCAM-1, MUC-1, MUC-16, L1-CAM, c-kit, NKG2D, NKG2D-Ligand, PD-L1, PD-L2, Lewis-V, CAW, CEA, c-MET, EGFR, EGFRvIII, Erb62, Her2, FAP, FR-a, EphA2, GD2, GD3, GPC3, IL-13Ra, Mesothelin, PSMA, PSCA, and VEGFR, preferably CD19 and/or CD20.

41. The method of any one of items 5 to 40, wherein said cell surface antigen is CD19.

42. The method of any one of items 5 to 41, wherein said cell surface antigen is CD20.

43. The method of any one of items 4 to 42, wherein step (A) comprises sub-steps of:
(A-I) Labeling said cell surface antigen on said cancer cells;
(A-II) Detecting said labelled cell surface antigen on said cancer cells by super-resolution microscopy; and
(A-III) Counting the number of labelled cell surface antigen molecules per cancer cell.

44. The method of any one of items 5 to 43, wherein said cell surface antigen is labelled in step (A) and step (A-I), respectively, by immunostaining.

45. The method of any one of items 1 to 44, wherein step (B) further comprises steps of:
(B-I) Classifying said cancer cell containing sample as positive for said cell surface antigen if the number of cell surface antigen molecules per cell obtained in step (A-III) is above a minimum threshold; and/or
(B-II) Classifying said cancer cell containing sample as negative for said cell surface antigen if the number of cell surface antigen molecules per cell obtained in step (A-III) is below a minimum threshold.

46. The method of item 45, wherein said minimum threshold is in the range of 4 to 300.

47. The method of item 45 or 46, wherein said minimum threshold is 4.

48. The method of item 45 or 46, wherein said minimum threshold is 8.

49. The method of item 45 or 46, wherein said minimum threshold is 16.

50. The method of item 45 or 46, wherein said minimum threshold is 32.

51. The method of item 45 or 46, wherein said minimum threshold is 64.

52. The method of item 45 or 46, wherein said minimum threshold is 100.

53. The method of item 45 or 46, wherein said minimum threshold is 200.

54. The method of item 45 or 46, wherein said minimum threshold is 300.

55. The method of any one of items 1 to 54, wherein based on the classification of said cancer cell-containing sample in step (B), a prediction on the eligibility of said patient for cancer therapy is made.

56. The method of item 55, wherein said patient is predicted to be eligible for cancer therapy if said classification of said cancer cell containing sample in step (B) for said cell surface antigen is positive.

57. The method of any one of items 1 to 56, wherein the method is a method for selecting a target antigen for cancer therapy.

58. The method of any one of items 1 to 57, wherein the method is a method for selecting a patient for cancer therapy.

59. The method of item 58, wherein said cancer therapy is cancer immunotherapy against said cell surface antigen.

60. The method of item 59, wherein said cancer immunotherapy is a targeted cancer immunotherapy against said cell surface antigen.

61. The method of item 60, wherein said targeted cancer immunotherapy is a cell-based targeted cancer immunotherapy against said cell surface antigen.

62. The method of item 61, wherein said cell-based targeted cancer immunotherapy is an immunotherapy against said cell surface antigen with chimeric antigen receptor (CAR)-engineered T-cells.

63. The method of any one of items 59 to 62, wherein said immunotherapy is an immunotherapy targeting a cell surface antigen selected from the group consisting of CD19, CD20, CD22, CD27, CD30, CD33, CD38, CD44v6, CD52, CD64, CD70, CD72, CD123, CD135, CD138, CD220, CD269, CD319, ROR1, ROR2, SLAMF7, BCMA, αvβ3-Integrin, α4β1-Integrin, EpCAM-1, MUC-1, MUC-16, L1-CAM, c-kit, NKG2D, NKG2D-Ligand, PD-L1, PD-L2, Lewis-Y, CAIX, CEA, c-MET, EGFR, EGFRvIll, ErbB2, Her2, FAP, FR-a, EphA2, GD2, GD3, GPC3, IL-13Ra, Mesothelin, PSMA, PSCA, VEGFR, preferably wherein said immunotherapy is an immunotherapy targeting CD19 and/or CD20.

64. The method of item 63, wherein said immunotherapy is an immunotherapy targeting CD19 and/or CD20.

65. The method of item 63, wherein said immunotherapy is an immunotherapy targeting CD19.

66. The method of item 63, wherein said immunotherapy is an immunotherapy targeting CD20.

67. The method of any one of items 1 to 66, wherein all the steps are of the method are carried out in vitro.

68. The method of any one of items 1 to 67, wherein the method does not comprise treatment of the human or animal body by surgery or therapy.

69. The method of any one of items 1 to 68, wherein the method is not a diagnostic method practiced on the human or animal body.

70. The method of any one of items 1 to 69, wherein said cancer cell-containing sample is a bone marrow aspirate.

71. The method of any one of items 1 to 70, wherein said cancer cell-containing sample comprises primary myeloma cells and the patient is a myeloma patient.

72. The method of any one of items 1 to 71, wherein said cancer cell-containing sample comprises primary myeloma cells expressing CD138 and the patient is a myeloma patient.

73. The method of any one of items 1 to 72, wherein said cancer cell containing sample is obtainable by positive selection of primary myeloblasts from bone marrow aspirate for CD138.

74. The method of item 73, wherein said selection is selection using magnetic beads.

75. An immune cell capable of targeting a cell surface antigen of a cell of a cancer, for use in a method for the treatment of said cancer in a patient, wherein in the method, the immune cell is to be administered to the patient.

76. The immune cell of item 75 for use of item 75, wherein said cancer is myeloma.

77. The immune cell of items 75 or 76 for use of items 75 or 76, wherein said cancer contains a fraction of cells positive for said cell surface antigen as determined according to any one of items 5 to 74.

78. The immune cell of any one of items 75 to 77 for the use of any one of items 75 to 77, wherein the method comprises cancer immunotherapy.

79. The immune cell of item 78 for the use of item 78, wherein said cancer immunotherapy is a targeted cancer immunotherapy.

80. The immune cell of item 79 for the use of item 79, wherein said targeted cancer immunotherapy is a cell-based targeted cancer immunotherapy.

81. The immune cell of item 79 or 80 for the use of item 79 or 80, wherein said targeted cancer immunotherapy is a targeted cancer immunotherapy targeting a cell surface antigen as defined in any one of items 63 to 66.

82. The immune cell of item 81 for the use of item 81, wherein the immune cell is capable of binding to said cell surface antigen.

83. The immune cell of any one of items 75 to 82 for the use of items 75 to 82, wherein the immune cell is capable of binding to CD19 and/or CD20.

84. The immune cell of any one of items 75 to 83 for the use of items 75 to 83, wherein the immune cell is capable of binding to CD20.

85. The immune cell of item 84 for the use of item 84, wherein the immune cell is capable of binding to the extracellular domain of CD20.

86. The immune cell of any one of items 75 to 85 for the use of items 75 to 85, wherein the immune cell is capable of binding to CD19.

87. The immune cell of item 86 for the use of item 86, wherein the immune cell is capable of binding to the extracellular domain of CD19.

88. The immune cell of any one of items 75 to 87 for use of items 75 to 87, wherein the cell is a cell expressing a chimeric antigen receptor.

89. The immune cell of item 88 for use of item 88, wherein the chimeric antigen receptor is capable of binding to said cell surface antigen.

90. The immune cell of item 88 or 89 for use of item 88 or 89, wherein the chimeric antigen receptor is capable of binding to CD19 and/or CD20.

91. The immune cell of any one of items 88 to 90 for use of items 88 to 90, wherein the chimeric antigen receptor is capable of binding to CD20.

92. The immune cell of any one of items 88 to 91 for use of items 88 to 91, wherein the chimeric antigen receptor is capable of binding to CD19.

93. The immune cell of any one of items 75 to 92 for use of any one of items 75 to 92, wherein the cell is a cell selected from the group of T cells, NK cells, and B cells.

94. The immune cell of any one of items 75 to 93 for use of any one of items 75 to 93, wherein the cell is a T cell.

95. The immune cell of any one of items 75 to 94 for the use of any one of items 75 to 94, wherein said cell-based targeted cancer immunotherapy is an immunotherapy with chimeric antigen receptor (CAR)-engineered T-cells.

96. The immune cell of any one of items 75 to 95 for the use of any one of items 75 to 95, wherein said patient is a patient eligible for said treatment as predictable by the method of any one of items 55 to 74.

97. The immune cell of any one of items 75 to 96 for the use of any one of items 75 to 96, wherein the cancer is negative for expression of said cell surface antigen as determined by flow cytometry.

98. The immune cell of item 97 for the use of item 97, wherein the cancer is positive for expression of said cell surface antigen as determined by super-resolution microscopy.

99. The immune cell of item 98 for the use of item 98, wherein the cancer is positive for expression of said cell surface antigen as determined by single-molecule localization microscopy.

100. The immune cell of items 98 or 99 for the use of items 98 or 99, wherein the cancer is positive for expression of said cell surface antigen as determined by dSTORM, STORM, PALM, or FPALM.

101. The immune cell of item 100 for the use of item 100, wherein the cancer is positive for expression of said cell surface antigen as determined by dSTORM.

102. The immune cell of any one of items 75 to 101 for the use of any one of items 75 to 101, wherein a fraction of the cancer cells expresses said cell surface antigen at a number of at least 4 cell surface antigen molecules per cell.

103. The immune cell of any one of items 75 to 101 for the use of any one of items 75 to 101, wherein a fraction of the cancer cells expresses said cell surface antigen at a number of at least 8 cell surface antigen molecules per cell.

104. The immune cell of any one of items 75 to 101 for the use of any one of items 75 to 101, wherein a fraction of the cancer cells expresses said cell surface antigen at a number of at least 16 cell surface antigen molecules per cell.

105. The immune cell of any one of items 75 to 101 for the use of any one of items 75 to 101, wherein a fraction of the cancer cells expresses said cell surface antigen at a number of at least 32 cell surface antigen molecules per cell.

106. The immune cell of any one of items 75 to 101 for the use of any one of items 75 to 101, wherein a fraction of the cancer cells expresses said cell surface antigen at a number of at least 64 cell surface antigen molecules per cell.

107. The immune cell of any one of items 75 to 101 for the use of any one of items 75 to 101, wherein a fraction of the cancer cells expresses said cell surface antigen at a number of at least 100 cell surface antigen molecules per cell.

108. The immune cell of any one of items 75 to 101 for the use of any one of items 75 to 101, wherein a fraction of the cancer cells expresses said cell surface antigen at a number of at least 200 cell surface antigen molecules per cell.

109. The immune cell of any one of items 75 to 101 for the use of any one of items 75 to 101, wherein a fraction of the cancer cells expresses said cell surface antigen at a number of at least 300 cell surface antigen molecules per cell.

110. The immune cell of any one of items 75 to 109 for the use of any one of items 75 to 109, wherein the cancer cells do not express said cell surface antigen at a number of more than 10,000 cell surface antigen molecules per cell.

111. The immune cell of any one of items 75 to 109 for the use of any one of items 75 to 109, wherein the cancer cells do not express said cell surface antigen at a number of more than 5,000 cell surface antigen molecules per cell.

112. The immune cell of any one of items 75 to 109 for the use of any one of items 75 to 109, wherein the cancer cells do not express said cell surface antigen at a number of more than 2,500 cell surface antigen molecules per cell.

113. The immune cell of any one of items 75 to 109 for the use of any one of items 75 to 109, wherein the cancer cells do not express said cell surface antigen at a number of more than 1,500 cell surface antigen molecules per cell.

114. The immune cell of any one of items 75 to 109 for the use of any one of items 75 to 109, wherein the cancer cells do not express said cell surface antigen at a number of more than 1,350 cell surface antigen molecules per cell.

115. The immune cell of any one of items 75 to 109 for the use of any one of items 75 to 109, wherein the cancer cells do not express said cell surface antigen at a number of more than 1,300 cell surface antigen molecules per cell.

116. The immune cell of any one of items 75 to 109 for the use of any one of items 75 to 109, wherein the cancer cells do not express said cell surface antigen at a number of more than 1,000 cell surface antigen molecules per cell.

117. The immune cell of any one of items 75 to 109 for the use of any one of items 75 to 109, wherein the cancer cells do not express said cell surface antigen at a number of more than 800 cell surface antigen molecules per cell.

118. The immune cell of any one of items 75 to 109 for the use of any one of items 75 to 109, wherein the cancer cells do not express said cell surface antigen at a number of more than 500 cell surface antigen molecules per cell.

119. The immune cell of any one of items 75 to 118 for the use of any one of items 75 to 118, wherein the treatment is a treatment in combination with myeloablative chemotherapy.

120. The immune cell of item 119 for the use of item 119, wherein the myeloablative chemotherapy comprises treatment with melphalan.

121. The immune cell of item 120 for the use of item 120, wherein melphalan at a dose between 100 mg per square meter and 200 mg per square meter, preferably wherein melphalan is to be administered at a dose of 140 mg per square meter.

122. The immune cell of any one of items 75 to 121 for the use of any one of items 75 to 121, wherein the treatment is a treatment in combination with autologous hematopoietic stem cell transplantation and/or wherein the treatment is a treatment in combination with allogeneic hematopoietic stem cell transplantation.

123. The immune cell of any one of items 88 to 122 for the use of items 88 to 122, wherein the chimeric antigen receptor is a chimeric antigen receptor having the amino acid sequence encoded by the nucleotide sequence of SEQ ID NO: 1 and/or SEQ ID NO: 3.

124. The immune cell of any one of items 88 to 122 for the use of items 88 to 122, wherein the chimeric antigen receptor is a chimeric antigen receptor having the amino acid sequence encoded by the nucleotide sequence of SEQ ID NO: 1.

125. The immune cell of any one of items 88 to 122 for the use of items 88 to 122, wherein the chimeric antigen receptor is a chimeric antigen receptor having the amino acid sequence encoded by the nucleotide sequence of SEQ ID NO: 3.

### BRIEF DESCRIPTION OF THE DRAWINGS

FIG. 1: Detection of CD19 on multiple myeloma cells using flow cytometry. Detection of primary myeloma cells by flow cytometry and dSTORM. CD138-purified bone marrow aspirates from multiple myeloma patients were stained with antibodies against CD138 and CD38 to detect myeloma cells and a CD19-specific antibody or corresponding isotype control. Examples are shown for (as judged by flow cytometry) highly CD19⁺ myeloma cells (A, patient M012), CD19⁺ MM cells (B, patient M016), ambiguous CD19 expression (C, patient M019) and CD19⁻ MM cells (D, patient M022). Gates were set on plasma cells (FSC/SSC) and CD138⁺/CD38⁺ MM cells.

Data for all patients are shown in Table 1 and FIG. 5.

FIG. 2: Detection of CD19 on multiple myeloma cells using dSTORM.

CD19 was detected on primary myeloma cells using conventional wide-field fluorescence (A) and dSTORM (B). Images depict CD19 molecules in the bottom plasma membrane (attached to glass surface) of a CD19⁺ (top row) and a CD19⁻ myeloma cell (bottom row). Small panels (C) display magnification of boxed regions revealing the markedly enhanced sensitivity of dSTORM. Fluorescence images of CD38 (D), CD138 (E) and the corresponding transmitted light image (F) for identification of the cells. Scale bars, 10 μm and 1 μm (C).

FIG. 3 (3A to 3D): Quantification of CD19 on multiple myeloma cells by dSTORM and eradication by CD19-CAR T-cells.

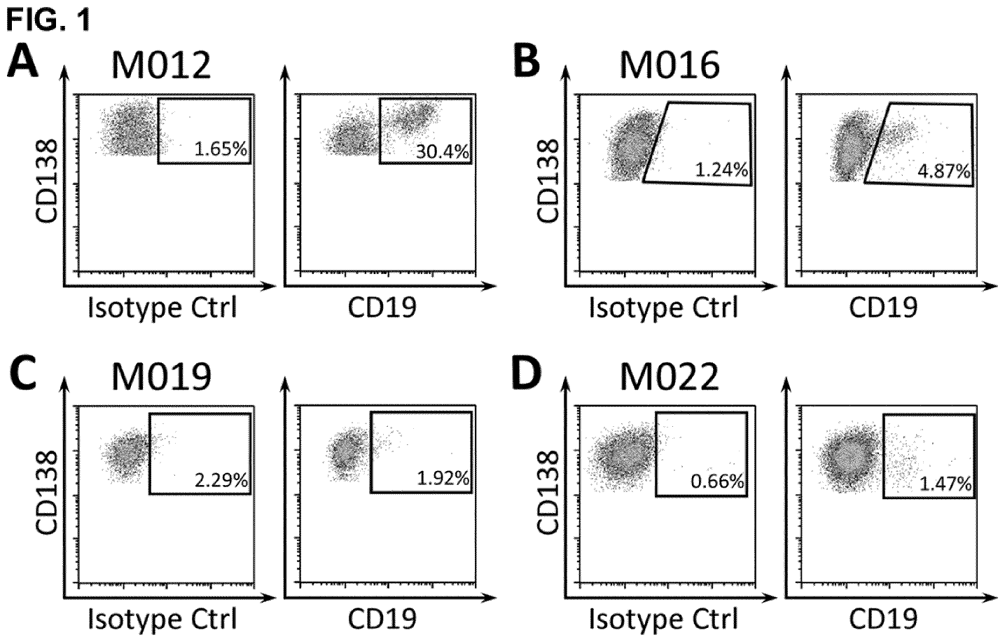

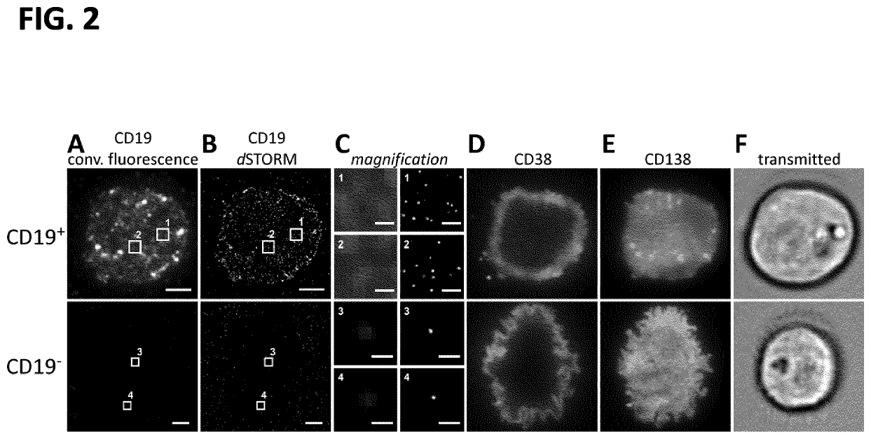

CD138-purified bone marrow aspirates from multiple myeloma patients were stained with antibodies against CD138 and CD38 to detect myeloma cells and a CD19-specific antibody or corresponding isotype control as indicated. The same patients as shown in FIG. 1 were investigated using dSTORM. Distribution of isotype antibody (first column) and CD19 (second column) densities in the plasma membranes of MM cells as quantified by dSTORM. The red segment of the distributions corresponds to the percentage of CD19-positive cells, as determined by flow cytometry measurements (see FIG. 1). Densities are given in logarithmic numbers of antibodies per μm². Density distributions were subsequently divided into a CD19-positive subpopulation (CD19-positive cells) and a CD19-negative subpopulation (CD19-negative cells). The latter group was defined by the density distribution pattern of the isotype control antibody (non-specific binding of the control antibody to the plasma membrane and glass surface). Distributions were fitted with a one or two log-normal function that was dependent on the fit accuracy calculated with an Anderson-Darling test (rejected at a p-value<0.05). Third and fourth column: logarithmic CD19 densities of CAR T-cell and control T-cell-treated MM cells. PDF: probability density function. Data for all patients are shown in Table 1 and FIG. 8.

FIG. 4: CD19 expression varies strongly among patients. (A) Mean protein densities on primary MM cells of CD19⁺ (dark gray) and CD19⁻ (light gray) subpopulations as measured by dSTORM. Displayed values are from one representative negative patient (M014) and from all CD19-positive patients, ranging from 0.2 (M017) to 3.1 (M022) CD19 molecules/μm². (B) Percentages of CD19⁺ and CD19⁻ cells, ranging from 10% (M022) to 80% (M019) of CD19-positive cells among patients.

FIG. 5 (FIGS. 5A & 5B): Detection of CD19 on multiple myeloma cells by flow cytometry CD138-purified bone marrow aspirates from multiple myeloma patients were stained with antibodies against CD138 and CD38 to detect myeloma cells (first line) and a CD19-specific antibody (third line) or a corresponding isotype control (second line) and measured by flow cytometry. Gates were set on plasma cells (FSC/SSC) and CD138⁺/CD38⁺ MM cells. Percentages indicated refer to CD19-positive cells within CD138⁺/CD38⁺ subset.

FIG. 6: dSTORM is 1000 times more sensitive than flow cytometry.

The CD38⁺/CD138⁺/CD19⁺ ALL cell line NALM-6 was stained with antibodies against CD138, CD38 and CD19 or the corresponding isotype control. (A) Flow cytornetric detection of CD19 on NALM-6 cells with decreasing dilutions of CD19-specific antibody (lower row) or corresponding isotype control (upper row). (B) Detection of CD19 antibody (black squares) and isotype control (red circles) by dSTORM. At a CD19 antibody concentration of 2.5 μg/ml (1:20 dilution), the CD19 density saturated at 3.4±0.2 CD19 antibodies/μm² (filled arrow). The lowest detectable density was 0.006±0.002 CD19 antibodies/μm², which was at 5×10⁻⁵ μg/ml (1:10⁶ dilution, open arrow). At an isotype antibody concentration of 5×10⁻⁵ μg/ml, it was not possible to detect any molecules (0 molecules/μm²), which is represented as a red open circle in the graph. The corresponding dSTORM images are depicted in (C), 2.5 μg/ml, and (D), 5×10⁻⁴ μg/ml CD19 antibody. Scale bars, 2 μm.

FIG. 7. Schematic illustration of CD19 classification. Density distributions were divided into a CD19-positive subpopulation (CD19-positive cells) and a CD19-negative subpopulation (CD19-negative cells; blue range). The latter group was defined by the density distribution pattern of the isotype control antibody (non-specific binding of the control antibody to the plasma membrane and glass surface). In this case, distributions were fitted to a two log-normal function, to estimate median (μ) values and to calculate density ranges from small (μ-2σ) to large (μ+2σ) values. The CD19-positive population was further divided into a CD19^{low} (orange range) and a CD19^{high} subpopulation (red range), depending on the cut-off value of 1,350 molecules per cell (see text for further details).

FIG. 8 (FIGS. 8A to 8K): Quantification of CD19 on multiple myeloma cells by dSTORM and eradication by CD19-CAR T-cells.

CD138-purified bone marrow aspirates from multiple myeloma patients were stained with antibodies against CD138 and CD38 to detect myeloma cells and a CD19-specific antibody or corresponding isotype control as indicated. Shown are distributions of all CD19-positive patients and one representative negative patient (D). Left panels: Logarithmic number of isotype and CD19 antibodies per μm² of untreated MM cells. Right panels: Logarithmic CD19 densities of control T-cell- and CAR T-cell-treated MM cells. Density distributions were subsequently divided into a CD19-positive subpopulation (CD19-positive cells) and a CD19-negative subpopulation (CD19-negative cells). The latter group was defined by the density distribution pattern of the isotype control antibody (non-specific binding of the control antibody to the plasma membrane and glass surface). Distributions were fitted with a one or two log-normal function that was dependent on the fit accuracy calculated with an Anderson-Darling test (rejected at a p-value<0.05. Effect of control T-cells was not evaluated for patient M008 (A). M014 (D) is an example of a completely CD19⁻ patient. PDF: probability density function. Data are also summarized in Table 1.

FIG. 9: CD19high and CD19low expression on primary multiple myeloma cells. (A, B) 4×4 μm sections of reconstructed dSTORM images showing single CD19 molecules in the surface-attached plasma membrane of immobilized MM cells. (A) Low expression of CD19 (13^{˜} molecules/cell, M017) and (B) high CD19 expression (^{˜}3000 molecules/cell, M022). Scale bars, 1 μm.

FIG. 10: Antigen-specific production of IFNy by CD19CAR T-cells upon cocultivation with primary MM cells. Un-transduced control CD8⁺ T-cells (black) or CD19CAR T-cells (light gray) were co-cultivated with primary myeloma cells or K562_CD19 at an effector:target ratio of 4:1 for 4 h in the presence of GolgiStop™. T-cells were treated with Cytofix/Cytoperm and stained for CD8 and IFNγ. Shown is the percentage of IFNγ⁺ T-cells in the presence of primary MM or K562_CD19 cells minus the percentage of IFNγ⁺ T-cells cultured for 4 h with medium only. Gates were set on lymphocytes (FSC/SSC), CD8⁺ and IFNγ⁺ cells. Every column represents a single experiment, except for K562_CD19 (n=12). ndt: cytokine production was not assessed for patient M020

FIG. 11. Specificity of CD19 antibody on control cell lines.

The used anti-CD19 antibody was tested for binding specificity by conventional wide-field microscopy (upper rows: normalized fluorescence, bottom rows: transmitted light). NALM-6 (A, B), MM.1S (C, D), K562 (E, F) and CD19 expressing K562_CD19 cells (G, H) were stained with Anti-CD19-AF647 antibody (column label: CD19) and its corresponding isotype-AF647 antibody (column label: Isotype). Scale bars, 7 μm.

FIG. 12. Detection of CD20 on myeloma cells by flow cytometry.

Flow cytometric analysis of CD20-expression on primary myeloma cells purified from bone marrow aspirates. Gating strategy for dot plots shown: FSC/SSC plasma cell gate→7-AAD⁻→CD138⁺/CD38⁺→lsotype control or CD20⁺.

FIG. 13: Quantification of CD20 on myeloma cells by dSTORM and elimination of CD20-positive myeloma cells by CD20CART.

(A) CD20 was detected on primary myeloma cells using conventional wide-field fluorescence and dSTORM. Images depict the bottom plasma membrane (attached to glass surface) of a CD20⁺ (upper row) or CD20⁻ myeloma cell (lower row). Shown are the transmitted light image, fluorescence images of CD38, CD138 for identification of the cells and CD20 molecules as detected by conventional fluorescence microsopy and dSTORM. Small panels display magnification of boxed regions revealing the markedly enhanced sensitivity of dSTORM. Scale bars, 1 μm and 0.2 μm.

(B) Quantification of CD20 using dSTORM.

CD138-purified bone marrow aspirates from 4 multiple myeloma patients were stained with antibodies against CD138 and CD38 to detect myeloma cells and a CD20-specific antibody or corresponding isotype control as indicated. Left panels: Logarithmic number of isotype and CD20 antibodies per μm² of untreated MM cells. Right panels: Logarithmic CD20 densities of control T-cell- and CAR T-cell-treated MM cells. Density distributions were subsequently divided into a CD20-positive subpopulation (CD20-positive cells) and a CD20-negative subpopulation (CD20-negative cells). The latter group was defined by the density distribution pattern of the isotype control antibody (non-specific binding of the control antibody to the plasma membrane and glass surface). Distributions were fitted with a one or two log-normal function that was dependent on the fit accuracy calculated with an Anderson-Darling test (rejected at a p-value<0.05). Panels depict merged data from 4 multiple myeloma patients. Fit of the isotype control is shown in all graphs for better comparisation (dotted line). Data for single patients are also summarized in Table 2 and depicted in FIG. 14.

(C) Representative 4×4 μm sections of reconstructed dSTORM images showing single CD20 molecules in the surface-attached plasma membrane of immobilized MM cells from 4 MM paptients. Scale bars, 1 μm.

FIG. 14: Quantification of CD20 on myeloma cells by dSTORM and elimination of CD20-positive myeloma cells by CD2OCART.

(A-D) CD138-purified bone marrow aspirates from 4 multiple myeloma patients were stained with antibodies against CD138 and CD38 to detect myeloma cells and a CD20-specific antibody or corresponding isotype control as indicated. Left panels: Logarithmic number of isotype and CD20 antibodies per μm² of untreated MM cells. Right panels: Logarithmic CD20 densities of control T-cell- and CAR T-cell-treated MM cells. Density distributions were subsequently divided into a CD20-positive subpopulation (CD20-positive cells) and a CD20-negative subpopulation (CD20-negative cells). The latter group was defined by the density distribution pattern of the isotype control antibody (non-specific binding of the control antibody to the plasma membrane and glass surface). Distributions were fitted with a one or two log-normal function that was dependent on the fit accuracy calculated with an Anderson-Darling test (rejected at a p-value<0.05). Effect of T-cells was not evaluated for patient M026 (B). Data are also summarized in Table 2.

### DETAILED DESCRIPTION OF THE INVENTION

CD19 is pursued as a target for CAR T-cell immunotherapy in MM. A recent study by Garfall et al reported complete remission in a myeloma patient who received CD19CART after myeloablative chemotherapy and autologous HSCT, even though only 0.05% of myeloma cells were CD19-positive as assessed by FC², but Garfall et al did not demonstrate any mechanism for this observation. The present inventors set out to test if CD19 is expressed on a higher proportion of myeloma cells than had been identified by FC in the study by Garfall et al, including whether there are myeloma cells that express CD19 at very low levels, which may, however, be sufficient for recognition by CD19CART^{2,13,14}. An obstacle to testing this hypothesis was the relatively high detection limit of FC, the prevailing detection method in clinical routine, with a detection limit in the order of few a thousands of molecules per Cell^{7,15,16}. In addition, the precise antigen threshold on tumor cells required to trigger and subsequently activate CART-cells has thus far been unknown. Several studies have attempted to extrapolate the lower detection limit of CARs with model cell lines, providing estimates in the range of hundreds of target molecules per Cell^{17,18}. However, these estimates have not been rigorously verified owing again to the lacking ability of FC to detect such low antigen levels on target cells.

Here, the inventors applied single-molecule sensitive super-resolution microscopy by dSTORM and show that in 10 out of 14 myeloma patients, CD19 is expressed on a large fraction of myeloma cells comprising up to 80% of the entire myeloma cell population. However, on the majority of myeloma cells, the expression level of CD19 is below the detection limit of FC and could only be visualized by dSTORM. The inventors also show that very low level expression of CD19 is sufficient for recognition and elimination by CD19CART and establish that the sensitivity threshold of CD19CART is far below 100 CD19 molecules per myeloma cell.

The inventors' data show that FC dramatically underestimates the percentage of myeloma cells that express CD19 and falsely classifies myeloma cells in 8 out of 10 patients as CD19-negative, even though CD19 is expressed on a fraction of myeloma cells at low levels as revealed by dSTORM imaging. The inventors' data suggest that myeloma cells that express less than 1,350 CD19 molecules are not detected by FC, which is consistent with previous reports on the sensitivity of this method in clinical routine¹⁵⁻¹⁸. The inventors show that in each of the 10 myeloma patients, where a proportion of CD19-expressing myeloma cells was detected (either at high or low density), these myeloma cells were readily eliminated after a short treatment with CD19CART in vitro. These data suggest that CD19CART might be effective against CD19-expressing myeloma cells in vivo. The CD19-CAR employed in the inventors' study has been validated in clinical trials in ALL and NHL^{3,4}. However, the inventors' data also show that in each of the 10 patients, there was a fraction of CD19-negative myeloma cells that were not eliminated by CDI9CART. These data suggest that complete responses of MM after CD19CART therapy may only be accomplished in conjunction with another effective antimyeloma treatment, e.g. melphalan (140 mg per square meter) as in the Garfall et al study. Indeed, recent studies with CD19CART in ALL and with B-cell maturation antigen (BCMA)-CART-cells in myeloma have shown that the presence of antigen-negative leukemia or myeloma cells leads to outgrowth of these cells and rapid relapse^{19,20}.

CARs are synthetic receptors and even though CD19CART have accomplished clinical approval in ALL and NHL, their mechanism of action is still a black box at the molecular level. A particular interest has been to determine the antigen sensitivity of CART-cells, both for predicting efficacy and for assessing safety. Here, the inventors provide for the first time direct evidence that CD19CART are able to recognize and eliminate myeloma cells that express less than 100 CD19 molecules on their surface. These data establish the sensitivity threshold for CART-cells and surpass predictions that have been made in previous studies with model tumor cell lines^{17,18}, but were limited by the inability of FC to enumerate antigens with single-molecule resolution. The inventors' data support the prior notion that CART-cells are more sensitive than conventional antibodies and bi-specific antibodies in detecting surface molecules on tumor cells¹⁷. Moreover, the inventors show that their findings of previously undetectable expression that is sufficient to trigger CART function are not limited to CD19 but also apply for additional antigens including CD20. Further, this study illustrates the challenge that CART-cells are more sensitive in detecting antigens on tumor cells than established analytical tools in clinical practice. Consequently, more sensitive detection methods than FC (and immunohistochemistry) need to be implemented into clinical routine in order to guide patient and antigen selection for CART-cells, and to detect low-level expression in healthy tissues to prevent toxicity. Efforts to implement dSTORM-analysis into clinical pathology are ongoing at the inventors' institution, but require further methodological simplification to become broadly applicable.

In summary, the inventors' data encourage the continued evaluation of CD19 as a target for CART-cells in MM. The inventors show that single-molecule sensitive fluorescence imaging methods such as dSTORM can aid in stratifying myeloma patients according to CD19 expression to identify patients who have the highest chance to benefit from this novel, highly innovative treatment. These insights are relevant not only for CD19CART in MM, but also for CART approaches targeting alternative antigens in other hematologic and solid tumor malignancies to exploit their full therapeutic potential and to ensure patient safety.

### Definitions and Embodiments

Unless otherwise defined below, the terms used in the present invention shall be understood in accordance with the common meaning known to the person skilled in the art.

Each publication, patent application, patent, and other reference cited herein is incorporated by reference in its entirety to the extent that it is not inconsistent with the present invention. References are indicated by their reference numbers and their corresponding reference details which are provided in the “references” section.

A targeting agent as described herein is an agent that, contrary to common medical agents, is capable of binding specifically to its target. A preferred targeting agent in accordance with the invention is capable of binding to CD19 on the cell surface, typically to the extracellular domain of CD19. Another preferred targeting agent in accordance with the invention is capable of binding to CD20 on the cell surface, typically to the extracellular domain of CD20.

In one embodiment of the invention, the targeting agent is capable of binding specifically to cancer cells expressing CD19 and/or CD20. In one embodiment of the invention, the targeting agent is capable of binding specifically to cancer cells expressing CD19. In one embodiment of the invention, the targeting agent is capable of binding specifically to cancer cells expressing CD20. In another embodiment of the invention, the targeting agent is capable of binding specifically to hematopoietic cells expressing CD19 and/or CD20. In another embodiment of the invention, the targeting agent is capable of binding specifically to hematopoietic cells expressing CD19. In another embodiment of the invention, the targeting agent is capable of binding specifically to hematopoietic cells expressing CD20. In another embodiment of the invention, the targeting agent is capable of binding specifically to hematopoietic cancer cells expressing CD19 and/or CD20. In another embodiment of the invention, the targeting agent is capable of binding specifically to hematopoietic cancer cells expressing CD19. In another embodiment of the invention, the targeting agent is capable of binding specifically to hematopoietic cancer cells expressing CD20. In a preferred embodiment of the invention, the targeting agent is capable of binding to primary myeloma cells expressing CD19 and/or CD20. In a preferred embodiment of the invention, the targeting agent is capable of binding to primary myeloma cells expressing CD19. In a preferred embodiment of the invention, the targeting agent is capable of binding to primary myeloma cells expressing CD20. In a preferred embodiment of the invention, the targeting agent is capable of binding to primary myeloma cells which express low levels of CD19 and/or CD20, preferably levels of CD19 and/or CD20 that cannot be detected by flow cytometry, more preferably low levels of CD19 and/or CD20 that cannot be detected by flow cytometry but can be detected by super-resolution microscopy, in particular single molecule localization microscopy (e.g. dSTORM). In another preferred embodiment of the invention, the targeting agent is capable of binding to primary myeloma cells which express low levels of CD20, preferably levels of CD20 that cannot be detected by flow cytometry, more preferably low levels of CD20 that cannot be detected by flow cytometry but can be detected by super-resolution microscopy, in particular single molecule localization microscopy (e.g. dSTORM). In a very preferred embodiment of the invention, the targeting agent is capable of binding to primary myeloma cells which express low levels of CD19, preferably levels of CD19 that cannot be detected by flow cytometry, more preferably low levels of CD19 that cannot be detected by flow cytometry but can be detected by super-resolution microscopy, in particular single molecule localization microscopy (e.g. dSTORM).

Preferably the immune cells and targeting agents as used herein are capable of causing a decrease in cancer cell number of the cancer cells expressing the target antigen. Preferably, this can be caused by cytotoxicity through necrosis or apoptosis, or this can be caused by inhibiting or stopping proliferation, i.e. inhibiting growth. This can be measured by various common methods and assays known in the art.

In one embodiment, the chimeric antigen receptor is capable of binding to CD19 and/or CD20. In one embodiment, the chimeric antigen receptor is capable of binding to CD19. In one embodiment, the chimeric antigen receptor is capable of binding to CD20. In a preferred embodiment, the chimeric antigen receptor is capable of binding to the extracellular domain of CD19 and/or CD20. In a preferred embodiment, the chimeric antigen receptor is capable of binding to the extracellular domain of CD19. In a preferred embodiment, the chimeric antigen receptor is capable of binding to the extracellular domain of CD20. In a preferred embodiment, the chimeric antigen receptor is expressed in immune cells, preferably T cells. In a preferred embodiment of the invention, the chimeric antigen receptor is expressed in T cells and allows said T cells to bind specifically to CD20- and/or CD19-expressing myeloma cells with high specificity to exert a growth inhibiting effect, preferably a cytotoxic effect, on said acute myeloid leukemia cells. In a preferred embodiment of the invention, the chimeric antigen receptor is expressed in T cells and allows said T cells to bind specifically to CD19-expressing myeloma cells with high specificity to exert a growth inhibiting effect, preferably a cytotoxic effect, on said acute myeloid leukemia cells. In a preferred embodiment of the invention, the chimeric antigen receptor is expressed in T cells and allows said T cells to bind specifically to CD20-expressing myeloma cells with high specificity to exert a growth inhibiting effect, preferably a cytotoxic effect, on said acute myeloid leukemia cells.

“Immunotherapy” as described herein refers to the transfer of immune cells into a patient for targeted treatment of cancer. The cells may have originated from the patient or from another individual. In immunotherapy, immune cells, preferably T cells, are typically extracted from an individual, preferably from the patient, genetically modified and cultured in vitro and administered to the patient. Immunotherapy is advantageous in that it allows targeted growth inhibiting, preferably cytotoxic, treatment of tumor cells without the non-targeted toxicity to non-tumor cells that occurs with conventional treatments.

In a preferred embodiment in accordance with the invention, T cells are isolated from a patient having multiple myeloma, transduced with a gene transfer vector encoding a chimeric antigen receptor capable of binding to CD19, and administered to the patient to treat multiple myeloma, preferably wherein the myeloma cells express CD19, more preferably low levels of CD19, more preferably low levels of CD19 that cannot be detected by flow cytometry, most preferably low levels of CD19 that cannot be detected by flow cytometry but can be detected by super-resolution microscopy, in particular single molecule localization microscopy (e.g. dSTORM). In a preferred embodiment, the T cells are CD8⁺ T cells or CD4⁺ T cells.

In another preferred embodiment in accordance with the invention, T cells are isolated from a patient having multiple myeloma, transduced with a gene transfer vector encoding a chimeric antigen receptor capable of binding to CD20, and administered to the patient to treat multiple myeloma, preferably wherein the myeloma cells express CD20, more preferably low levels of CD20, more preferably low levels of CD20 that cannot be detected by flow cytometry, most preferably low levels of CD20 that cannot be detected by flow cytometry but can be detected by super-resolution microscopy, in particular single molecule localization microscopy (e.g. dSTORM). In a preferred embodiment, the T cells are CD8⁺ T cells or CD4⁺ T cells.

In another preferred embodiment in accordance with the invention, T cells are isolated from a patient having multiple myeloma, transduced with a gene transfer vector encoding a chimeric antigen receptor capable of binding to CD19 and/or CD20, and administered to the patient to treat multiple myeloma, preferably wherein the myeloma cells express CD19 and/or CD20, more preferably low levels of CD19 and/or CD20, more preferably low levels of CD19 and/or CD20 that cannot be detected by flow cytometry, most preferably low levels of CD19 and/or CD20 that cannot be detected by flow cytometry but can be detected by super-resolution microscopy, in particular single molecule localization microscopy (e.g. dSTORM). In a preferred embodiment, the T cells are CD8⁺ T cells or CD4⁺ T cells.

Terms such as “treatment of cancer” or “treating cancer” according to the present invention refer to a therapeutic treatment. An assessment of whether or not a therapeutic treatment works can, for instance, be made by assessing whether the treatment inhibits cancer growth in the treated patient or patients. Preferably, the inhibition is statistically significant as assessed by appropriate statistical tests which are known in the art. Inhibition of cancer growth may be assessed by comparing cancer growth in a group of patients treated in accordance with the present invention to a control group of untreated patients, or by comparing a group of patients that receive a standard cancer treatment of the art plus a treatment according to the invention with a control group of patients that only receive a standard cancer treatment of the art. Such studies for assessing the inhibition of cancer growth are designed in accordance with accepted standards for clinical studies, e.g. double-blinded, randomized studies with sufficient statistical power. The term “treating cancer” includes an inhibition of cancer growth where the cancer growth is inhibited partially (i.e. where the cancer growth in the patient is delayed compared to the control group of patients), an inhibition where the cancer growth is inhibited completely (i.e. where the cancer growth in the patient is stopped), and an inhibition where cancer growth is reversed (i.e. the cancer shrinks). An assessment of whether or not a therapeutic treatment works can be made based on known clinical indicators of cancer progression.

A treatment of cancer according to the present invention does not exclude that additional or secondary therapeutic benefits also occur in patients. However, it is understood that the primary treatment for which protection is sought is for treating the cancer itself, and any secondary or additional effects only reflect optional, additional advantages of the treatment of cancer growth.

The treatment of cancer according to the invention can be a first-line therapy, a second-line therapy, a third-line therapy, or a fourth-line therapy. The treatment can also be a therapy that is beyond is beyond fourth-line therapy. The meaning of these terms is known in the art and in accordance with the terminology that is commonly used by the US National Cancer Institute.

The term “capable of binding” as used herein refers to the capability to form a complex with a molecule that is to be bound (e.g. CD19 and/or CD20). Binding typically occurs non-covalently by intermolecular forces, such as ionic bonds, hydrogen bonds and Van der Waals forces and is typically reversible. Various methods and assays to determine binding capability are known in the art. Binding is usually a binding with high affinity, wherein the affinity as measured in K_{D} values is preferably is less than 1 μM, more preferably less than 100 nM, even more preferably less than 10 nM, even more preferably less than 1 nM, even more preferably less than 100 pM, even more preferably less than 10 pM, even more preferably less than 1 pM.

As used herein, each occurrence of terms such as “comprising” or “comprises” may optionally be substituted with “consisting of” or “consists of”.

A pharmaceutically acceptable carrier, including any suitable diluent or, can be used herein as known in the art. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. Pharmaceutically acceptable carriers include, but are not limited to, saline, buffered saline, dextrose, water, glycerol, sterile isotonic aqueous buffer, and combinations thereof. It will be understood that the formulation will be appropriately adapted to suit the mode of administration.

Compositions and formulations in accordance with the present invention are prepared in accordance with known standards for the preparation of pharmaceutical compositions and formulations. For instance, the compositions and formulations are prepared in a way that they can be stored and administered appropriately, e.g. by using pharmaceutically acceptable components such as carriers, excipients or stabilizers. Such pharmaceutically acceptable components are not toxic in the amounts used when administering the pharmaceutical composition or formulation to a patient. The pharmaceutical acceptable components added to the pharmaceutical compositions or formulations may depend on the chemical nature of the inhibitor and targeting agent present in the composition or formulation (depend on whether the targeting agent is e.g. an antibody or fragment thereof or a cell expressing a chimeric antigen receptor), the particular intended use of the pharmaceutical compositions and the route of administration.

The “number of cell surface antigen molecules per cell” as referred to herein can be any such number in accordance with the meaning of the term that is known in the art. In a non-limiting preferred embodiment, the “number of cell surface antigen molecules per cell” is an average number of molecules per cell with respect to the cells or the fraction of cells expressing said cell surface antigen. In a more preferred non-limiting embodiment, the “number of cell surface antigen molecules per cell” is the mean value of the number of molecules per cell with respect to the cells or the fraction of cells expressing said cell surface antigen. In another non-limiting embodiment, the “number of cell surface antigen molecules per cell” is the median number of molecules per cell with respect to the cells or the fraction of cells expressing said cell surface antigen.

Any numbers of molecules of cell surface antigens as referred to herein can be determined by suitable methods. Preferably, these numbers can be determined by super-resolution microscopy, more preferably by single-molecule localization microscopy such as dSTORM, STORM, PALM, or PALM, and most preferably by dSTORM.

It is understood that for any methods of the invention using immunostaining, including the above-mentioned super-resolution microscopy methods such as dSTORM which can use immunostaining, any antibodies used therein are used at a suitable concentration. In a preferred embodiment, the antibodies used in the invention are used at saturating conditions. In another preferred embodiment, the methods of the invention are performed under conditions that have been clinically validated.

In a preferred embodiment in accordance with the invention, the composition or formulation is suitable for administration to humans, preferably the formulation is sterile and/or non-pyrogenic.

### EXAMPLES

Additional aspects and details of the invention are exemplified by the following non-limiting examples.

Immunotherapy with chimeric antigen receptor (CAR)-engineered T-cells targeting CD19 (CD19CART) was investigated in multiple myeloma, a clonal proliferation of plasma cells. A recent study by Garfall et al reported complete remission in a myeloma patient who received CD19CART after myeloablative chemotherapy and autologous stem cell transplantation, even though only 0.05% of myeloma cells expressed CD19 by flow cytometry (FC), the routine clinical detection method. The study sparked controversy over the role of CDI9CART for treating myeloma.

The inventors generated expression profiles of CD19 on myeloma cells from n=14 patients by single-molecule sensitive direct stochastic optical reconstruction microscopy (dSTORM), and compared them to profiles obtained by FC. In parallel, myeloma cells were treated with CD19CART in vitro.

In 10 out of 14 patients, the inventors detected CD19 on a fraction of myeloma cells (range: 10.3%-80%) by dSTORM. The majority of myeloma cells expressed CD19 at very low levels, below the detection limit of FC. FC detected CD19 only in 2 of these 10 patients on a smaller fraction of cells (range: 4.9%-30.4%). Four patients were CD19-negative by dSTORM. Treatment with CD19CART led to elimination of myeloma cells, even when CD19 was undetectable by FC. In a subset of patients, CD19 is expressed on a large fraction of myeloma cells, but remains undetected by FC. These patients are candidates for CD19CART cell therapy. The inventors demonstrate that that the threshold for CDI9CART recognition is far below 100 CD19 molecules per target cell, surpassing previous assumptions on the sensitivity of this novel treatment.

In particular, the Examples were carried out as follows:

### Human Subjects

Bone marrow aspirates were obtained from patients with multiple myeloma, and T-cells for CAR-modification were isolated from the peripheral blood of healthy donors. All participants provided written informed consent to participate in research protocols approved by the institutional review board of the University of Würzburg.

### Flow Cytometry Analyses

Primary myeloma cells were isolated from bone marrow using positive selection with anti-CD138 magnetic beads (Miltenyi, Bergisch-Gladbach, Germany) and stained with anti-CD19AF647 (clone: HIB19), CD20AF647 (clone: 2H7) or AF647 isotype control and anti-CD38-AF488, anti-CD138-PE antibodies, and 7-AAD to exclude dead cells from analysis.

### CAR T-Cell Treatment

Myeloma cells (2.5×10⁴) were co-cultured with CD19CART, CD2OCART (1×10⁵) or control untransduced T-cells (1×10⁵) for 4 hours in 96-well round-bottom plates prior to dSTORM-analysis. The CD19-CAR has been described²¹.

### dSTORM-Analyses

Myeloma cells were stained with anti-CD19-AF647, anti-CD20-AF647, anti-CD38-AF488 and anti-CD138-AF555 antibodies or AF647 isotype control antibodies (BioLegend, London, United Kingdom). Images were acquired on an Olympus IX-71 inverted microscope, dSTORM images were reconstructed using the single-molecule localization software rapidSTORM3.3²² and quantification of CD19 was performed using a custom script written with Mathematica (WolframResearch, Inc., Mathematica, Version 11.2, Champaign, Ill.).

### Primary Myeloma Cells

Bone marrow aspirate was diluted 1:10 in phosphate-buffered saline (PBS), and leukocytes were isolated using Ficoll-hypaque density centrifugation in 50 mL LeukoSep tubes (Greiner Bio One, Frickenhausen, Germany). CD138⁺ myeloma cells were isolated using positive selection with CD138-MicroBeads (Miltenyi, Bergisch-Gladbach, Germany).

### Cell Lines and Cell Culture Media

NALM-6 (DSMZ, Heidelberg, Germany), MM.1S and K562 (both ATCC, Manassas, Va., USA) cells were maintained in RPMI-1640 medium containing 8% fetal calf serum (FCS), 2 mM L-glutamine, and 100 U/mL penicillin/streptomycin (all components from Gibco, Thermo Scientific, Schwerte, Germany). K562_CD19 cells were generated by lentiviral transduction with human CD19. Primary myeloma cells and T-cells were maintained in RPMI-1640 medium containing 8% human serum, 2 mM Glutamax, 0,.% β-mercaptoethanol and 100 U/mL penicillin/streptomycin (T-cell medium; all other components from Gibco). T-cell cultures were supplemented with 50 U/mI IL-2 (Proleukin, Novartis, Basel, Switzerland).

### Generation of CD19CART and CD20CART

The vector design and experimental procedure has been described in a previous study²¹. In brief, peripheral blood mononuclear cells (PBMCs) of healthy donors were purified using Ficoll-hypaque density centrifugation in 50 mL LeukoSep tubes (Greiner Bio One), and CD8⁺ T-cells were isolated using negative magnetic sorting (CD8⁺ T-cell Isolation Kit, human, Miltenyi). T-cells were stimulated with anti-CD3/CD28 magnetic beads (Dynabeads® Human T-Activator CD3/CD28, ThermoScientific) and transduced with an epHIV7 lentivirus encoding a CAR construct comprising the following: an anti-CD19 or -CD20 single chain variable fragment derived from FMC63 and Leu16, respectively; an IgG4-Fc hinge spacer; a CD28 transmembrane region; a 4-1BB_CD3ζ signaling module; and a truncated epidermal growth factor receptor (EGFR) transduction marker²³. T-cells were enriched for EGFRt⁺ using the biotinylated anti-EGFR monoclonal antibody (mAb) Cetuximab (Merck, Darmstadt, Germany) and anti-Biotin Microbeads (Miltenyi). Purified CD19CART, CD2OCART and non-transduced control T-cells were expanded with irradiated CD19⁺/CD20⁺ feeder cells as previously described²⁴ and stored in aliquots in liquid nitrogen until functional testing.

Methods to generate chimeric antigen receptors, chimeric antigen receptor-expressing vectors, and methods for transducing said vectors are known in the art. Non-limiting exemplary methods include those described previously^{25,26}, which are incorporated herein by reference in their entirety for all purposes.

In a preferred embodiment of the invention, the chimeric antigen receptor is a CD19 CAR having the amino acid sequence encoded by the nucleotide sequence of SEQ ID NO: 1. In a more preferred embodiment, the CD19 CAR having the amino acid sequence encoded by SEQ ID NO: 1. can be expressed using the lentiviral vector having the nucleotide sequence of SEQ ID NO: 2.

In a preferred embodiment of the invention, the chimeric antigen receptor is a CD20 CAR having the amino acid sequence encoded by the nucleotide sequence of SEQ ID NO: 3. In a more preferred embodiment, the CD20 CAR having the amino acid sequence encoded by SEQ ID NO: 3 can be expressed using the lentiviral vector having the nucleotide sequence of SEQ ID NO: 4.

(nucleotide sequence encoding the CD19 CAR)
SEQ ID NO: 1
atgctgctgctggtgaccagcctgctgctgtgcgagctgccccaccccgcctttctgctgatccccgacatccagatgacccaga
ccacctccagcctgagcgccagcctgggcgaccgggtgaccatcagctgccgggccagccaggacatcagcaagtacctgaactg
gtatcagcagaagcccgacggcaccgtcaagctgctgatctaccacaccagccggctgcacagcggcgtgcccagccggtttagc
ggcagcggctccggcaccgactacagcctgaccatctccaacctggaacaggaagatatcgccacctacttttgccagcagggca
acacactgccctacacctttggcggcggaacaaagctggaaatcaccggcagcacctccggcagcggcaagcctggcagcggcga
gggcagcaccaagggcgaggtgaagctgcaggaaagcggccctggcctggtggcccccagccagagcctgagcgtgacctgcacc
gtgagcggcgtgagcctgcccgactacggcgtgagctggatccggcagccccccaggaagggcctggaatggctgggcgtgatct
ggggcagcgagaccacctactacaacagcgccctgaagagccggctgaccatcatcaaggacaacagcaagagccaggtgttcct
gaagatgaacagcctgcagaccgacgacaccgccatctactactgcgccaagcactactactacggcggcagctacgccatggac
tactggggccagggcaccagcgtgaccgtgagcagcgaatctaagtacggaccgccctgccccccttgccctatgttctgggtgc
tggtggtggtcggaggcgtgctggcctgctacagcctgctggtcaccgtggccttcatcatcttttgggtgaaacggggcagaaa
gaaactcctgtatatattcaaacaaccatttatgagaccagtacaaactactcaagaggaagatggctgtagctgccgatttcca
gaagaagaagaaggaggatgtgaactgcgggtgaaggttcagcagaagcgccgacgcccctgcctaccagcagggccagaatcag
ctgtacaacgagctgaacctgggcagaagggaagagtacgacgtcctggataagcggagaggccgggaccctgagatgggcggca
agcctcggcggaagaacccccaggaaggcctgtataacgaactgcagaaagacaagatggccgaggcctacagcgagatcggcat
gaagggcgagcggaggcggggcaagggccacgacggcctgtatcagggcctgtccaccgccaccaaggatacctacgacgccctg
cacatgcaggccctgcccccaaggctcgagggcggcggagagggcagaggaagtcttctaacatgcggtgacgtggaggagaatc
ccggccctaggatgcttctcctggtgacaagccttctgctctgtgagttaccacacccagcattcctcctgatcccacgcaaagt
gtgtaacggaataggtattggtgaatttaaagactcactctccataaatgctacgaatattaaacacttcaaaaactgcacctcc
atcagtggcgatctccacatcctgccggtggcatttaggggtgactccttcacacatactcctcctctggatccacaggaactgg
atattctgaaaaccgtaaaggaaatcacagggtttttgctgattcaggcttggcctgaaaacaggacggacctccatgcctttga
gaacctagaaatcatacgcggcaggaccaagcaacatggtcagttttctcttgcagtcgtcagcctgaacataacatccttggga
ttacgctccctcaaggagataagtgatggagatgtgataatttcaggaaacaaaaatttgtgctatgcaaatacaataaactgga
aaaaactgtttgggacctccggtcagaaaaccaaaattataagcaacagaggtgaaaacagctgcaaggccacaggccaggtctg
ccatgccttgtgctcccccgagggctgctggggcccggagcccagggactgcgtctcttgccggaatgtcagccgaggcagggaa
tgcgtggacaagtgcaaccttctggagggtgagccaagggagtttgtggagaactctgagtgcatacagtgccacccagagtgcc
tgcctcaggccatgaacatcacctgcacaggacggggaccagacaactgtatccagtgtgcccactacattgacggcccccactg
cgtcaagacctgcccggcaggagtcatgggagaaaacaacaccctggtctggaagtacgcagacgccggccatgtgtgccacctg
tgccatccaaactgcacctacggatgcactgggccaggtcttgaaggctgtccaacgaatgggcctaagatcccgtccatcgcca
ctgggatggtgggggccctcctcttgctgctggtggtggccctggggatcggcctcttcatgtga
(nucleotide sequence representing the expression vector encoding the CD19 CAR)
SEQ ID NO: 2
gttagaccagatctgagcctgggagctctctggctaactagggaacccactgcttaagcctcaataaagcttgccttgagtgctt
caagtagtgtgtgcccgtctgttgtgtgactctggtaactagagatccctcagacccttttagtcagtgtggaaaatctctagca
tggcgcccgaacagggacttgaaagcgaaagggaaaccagaggagctctctcgacgcaggactcggcttgctgaagcgcgcacgg
caagaggcgaggggcggcgactggtgagtacgccaaaaattttgactagcggaggctagaaggagagagatgggtgcgagagcgt
cagtattaagcgggggagaattagatcgatgggaaaaaattcggttaaggccagggggaaagaaaaaatataaattaaaacatat
agtatgggcaagcagggagctagaacgattcgcagttaatcctggcctgttagaaacatcagaaggctgtagacaaatactggga
cagctacaaccatcccttcagacaggatcagaagaacttagatcattatataatacagtagcaaccctctattgtgtgcatcaaa
ggatagagataaaagacaccaaggaagctttagacaagatagaggaagagcaaaacaaaagtaagaaaaaagcacagcaagcagc
agctgacacaggacacagcaatcaggtcagccaaaattaccctatagtgcagaacatccaggggcaaatggtacatcaggccata
tcacctagaactttaaatgcatgggtaaaagtagtagaagagaaggctttcagcccagaagtgatacccatgttttcagcattat
cagaaggagccaccccacaagatttaaacaccatgctaaacacagtggggggacatcaagcagccatgcaaatgttaaaagagac
catcaatgaggaagctgcaggcaaagagaagagtggtgcagagagaaaaaagagcagtgggaataggagctttgttccttgggtt
cttgggagcagcaggaagcactatgggcgcagcgtcaatgacgctgacggtacaggccagacaattattgtctggtatagtgcag
cagcagaacaatttgctgagggctattgaggcgcaacagcatctgttgcaactcacagtctggggcatcaagcagctccaggcaa
gaatcctggctgtggaaagatacctaaaggatcaacagctcctggggatttggggttgctctggaaaactcatttgcaccactgc
tgtgccttggatctacaaatggcagtattcatccacaattttaaaagaaaaggggggattggggggtacagtgcaggggaaagaa
tagtagacataatagcaacagacatacaaactaaagaattacaaaaacaaattacaaaaattcaaaattttcgggtttattacag
ggacagcagagatccagtttggggatcaattgcatgaagaatctgcttagggttaggcgttttgcgctgcttcgcgaggatctgc
gatcgctccggtgcccgtcagtgggcagagcgcacatcgcccacagtccccgagaagttggggggaggggtcggcaattgaaccg
gtgcctagagaaggtggcgcggggtaaactgggaaagtgatgtcgtgtactggctccgcctttttcccgagggtgggggagaacc
gtatataagtgcagtagtcgccgtgaacgttctttttcgcaacgggtttgccgccagaacacagctgaagcttcgaggggctcgc
atctctccttcacgcgcccgccgccctacctgaggccgccatccacgccggttgagtcgcgttctgccgcctcccgcctgtggtg
cctcctgaactgcgtccgccgtctaggtaagtttaaagctcaggtcgagaccgggcctttgtccggcgctcccttggagcctacc
tagactcagccggctctccacgctttgcctgaccctgcttgctcaactctacgtctttgtttcgttttctgttctgcgccgttac
agatccaagctgtgaccggcgcctacggctagcgccgccaccatgctgctgctggtgaccagcctgctgctgtgcgagctgcccc
accccgcctttctgctgatccccgacatccagatgacccagaccacctccagcctgagcgccagcctgggcgaccgggtgaccat
cagctgccgggccagccaggacatcagcaagtacctgaactggtatcagcagaagcccgacggcaccgtcaagctgctgatctac
cacaccagccggctgcacagcggcgtgcccagccggtttagcggcagcggctccggcaccgactacagcctgaccatctccaacc
tggaacaggaagatatcgccacctacttttgccagcagggcaacacactgccctacacctttggcggcggaacaaagctggaaat
caccggcagcacctccggcagcggcaagcctggcagcggcgagggcagcaccaagggcgaggtgaagctgcaggaaagcggccct
ggcctggtggcccccagccagagcctgagcgtgacctgcaccgtgagcggcgtgagcctgcccgactacggcgtgagctggatcc
ggcagccccccaggaagggcctggaatggctgggcgtgatctggggcagcgagaccacctactacaacagcgccctgaagagccg
gctgaccatcatcaaggacaacagcaagagccaggtgttcctgaagatgaacagcctgcagaccgacgacaccgccatctactac
tgcgccaagcactactactacggcggcagctacgccatggactactggggccagggcaccagcgtgaccgtgagcagcgaatcta
agtacggaccgccctgccccccttgccctatgttctgggtgctggtggtggtcggaggcgtgctggcctgctacagcctgctggt
caccgtggccttcatcatcttttgggtgaaacggggcagaaagaaactcctgtatatattcaaacaaccatttatgagaccagta
caaactactcaagaggaagatggctgtagctgccgatttccagaagaagaagaaggaggatgtgaactgcgggtgaagttcagca
gaagcgccgacgcccctgcctaccagcagggccagaatcagctgtacaacgagctgaacctgggcagaagggaagagtacgacgt
cctggataagcggagaggccgggaccctgagatgggcggcaagcctcggcggaagaacccccaggaaggcctgtataacgaactg
cagaaagacaagatggccgaggcctacagcgagatcggcatgaagggcgagcggaggcggggcaagggccacgacggcctgtatc
agggcctgtccaccgccaccaaggatacctacgacgccctgcacatgcaggccctgcccccaaggctcgagggcggcggagaggg
cagaggaagtcttctaacatgcggtgacgtggaggagaatcccggccctaggatgcttctcctggtgacaagccttctgctctgt
gagttaccacacccagcattcctcctgatcccacgcaaagtgtgtaacggaataggtattggtgaatttaaagactcactctcca
taaatgctacgaatattaaacacttcaaaaactgcacctccatcagtggcgatctccacatcctgccggtggcatttaggggtga
ctccttcacacatactcctcctctggatccacaggaactggatattctgaaaaccgtaaaggaaatcacagggtttttgctgatt
caggcttggcctgaaaacaggacggacctccatgcctttgagaacctagaaatcatacgcggcaggaccaagcaacatggtcagt
tttctcttgcagtcgtcagcctgaacataacatccttgggattacgctccctcaaggagataagtgatggagatgtgataatttc
aggaaacaaaaatttgtgctatgcaaatacaataaactggaaaaaactgtttgggacctccggtcagaaaaccaaaattataagc
aacagaggtgaaaacagctgcaaggccacaggccaggtctgccatgccttgtgctcccccgagggctgctggggcccggagccca
gggactgcgtctcttgccggaatgtcagccgaggcagggaatgcgtggacaagtgcaaccttctggagggtgagccaagggagtt
tgtggagaactctgagtgcatacagtgccacccagagtgcctgcctcaggccatgaacatcacctcacaggacggggaccagaca
actgtatccagtgtgcccactacattgacggcccccactgcgtcaagacctgcccggcaggagtcatgggagaaaacaacaccct
ggtctggaagtacgcagacgccggccatgtgtgccacctgtgccatccaaactgcacctacggatgcactgggccaggtcttgaa
ggctgtccaacgaatgggcctaagatcccgtccatcgccactgggatggtgggggccctcctcttgctgctggtggtggccctgg
ggatcggcctcttcatgtgagcggccgctctagacccgggctgcaggaattcgatatcaagcttatcgataatcaacctctggat
tacaaaatttgtgaaagattgactggtattcttaactatgttgctccttttacgctatgtggatacgctgctttaatgcctttgt
atcatgctattgcttcccgtatggctttcattttctcctccttgtataaatcctggttgctgtctctttatgaggagttgtggcc
cgttgtcaggcaacgtggcgtggtgtgcactgtgtttgctgacgcaacccccactggttggggcattgccaccacctgtcagctc
ctttccgggactttcgctttccccctccctattgccacggcggaactcatcgccgcctgccttgcccgctgctggacaggggctc
ggctgttgggcactgacaattccgtggtgttgtcggggaaatcatcgtcctttccttggctgctcgcctgtgttgccacctggat
tctgcgcgggacgtccttctgctacgtcccttcggccctcaatccagcggaccttccttcccgcggcctgctgccggctctgcgg
cctcttccgcgtcttcgccttcgccctcagacgagtcggatctccctttgggccgcctccccgcatcgataccgtcgactagccg
tacctttaagaccaatgacttacaaggcagctgtagatcttagccactttttaaaagaaaaggggggactggaagggctaattca
ctcccaaagaagacaagatctgctttttgcctgtactgggtctctctggttagaccagatctgagcctgggagctctctggctaa
ctagggaacccactgcttaagcctcaataaagcttgccttgagtgcttcaagtagtgtgtgcccgtctgttgtgtgactctggta
actagagatccctcagacccttttagtcagtgtggaaaatctctagcagaattcgatatcaagcttatcgataccgtcgacctcg
agggggggcccggtacccaattcgccctatagtgagtcgtattacaattcactggccgtcgttttacaacgtcgtgactgggaaa
accctggcgttacccaacttaatcgccttgcagcacatccccctttcgccagctggcgtaatagcgaagaggcccgcaccgatcg
cccttcccaacagttgcgcagcctgaatggcgaatggaaattgtaagcgttaatattttgttaaaattcgcgttaaatttttgtt
aaatcagctcattttttaaccaataggccgaaatcggcaaaatcccttataaatcaaaagaatagaccgagatagggttgagtgt
tgttccagtttggaacaagagtccactattaaagaacgtggactccaacgtcaaagggcgaaaaaccgtctatcagggcgatggc
ccactacgtgaaccatcaccctaatcaagttttttggggtcgaggtgccgtaaagcactaaatcggaaccctaaagggagccccc
gatttagagcttgacggggaaagccggcgaacgtggcgagaaaggaagggaagaaagcgaaaggagcgggcgctagggcgctggc
aagtgtagcggtcacgctgcgcgtaaccaccacacccgccgcgcttaatgcgccgctacagggcgcgtcaggtggcacttttcgg
ggaaatgtgcgcggaacccctatttgtttatttttctaaatacattcaaatatgtatccgctcatgagacaataaccctgataaa
tgcttcaataatattgaaaaaggaagagtatgagtattcaacatttccgtgtcgcccttattcccttttttgcggcattttgcct
tcctgtttttgctcacccagaaacgctggtgaaagtaaaagatgctgaagatcagttgggtgcacgagtgggttacatcgaactg
gatctcaacagcggtaagatccttgagagttttcgccccgaagaacgttttccaatgatgagcacttttaaagttctgctatgtg
gcgcggtattatcccgtattgacgccgggcaagagcaactcggtcgccgcatacactattctcagaatgacttggttgagtactc
accagtcacagaaaagcatcttacggatggcatgacagtaagagaattatgcagtgctgccataaccatgagtgataacactgcg
gccaacttacttctgacaacgatcggaggaccgaaggagctaaccgcttttttgcacaacatgggggatcatgtaactcgccttg
atcgttgggaaccggagctgaatgaagccataccaaacgacgagcgtgacaccacgatgcctgtagcaatggcaacaacgttgcg
caaactattaactggcgaactacttactctagcttcccggcaacaattaatagactggatggaggcggataaagttgcaggacca
cttctgcgctcggcccttccggctggctggtttattgctgataaatctggagccggtgagcgtgggtctcgcggtatcattgcag
cactggggccagatggtaagccctcccgtatcgtagttatctacacgacggggagtcaggcaactatggatgaacgaaatagaca
gatcgctgagataggtgcctcactgattaagcattggtaactgtcagaccaagtttactcatatatactttagattgatttaaaa
cttcatttttaatttaaaaggatctaggtgaagatcctttttgataatctcatgaccaaaatcccttaacgtgagttttcgttcc
actgagcgtcagaccccgtagaaaagatcaaaggatcttcttgagatcctttttttctgcgcgtaatctgctgcttgcaaacaaa
aaaaccaccgctaccagcggtggtttgtttgccggatcaagagctaccaactctttttccgaaggtaactggcttcagcagagcg
cagataccaaatactgttcttctagtgtagccgtagttaggccaccacttcaagaactctgtagcaccgcctacatacctcgctc
tgctaatcctgttaccagtggctgctgccagtggcgataagtcgtgtcttaccgggttggactcaagacgatagttaccggataa
ggcgcagcggtcgggctgaacggggggttcgtgcacacagcccagcttggagcgaacgacctacaccgaactgagatacctacag
cgtgagctatgagaaagcgccacgcttcccgaagggagaaaggcggacaggtatccggtaagcggcagggtcggaacaggagagc
gcacgagggagcttccagggggaaacgcctggtatctttatagtcctgtcgggtttcgccacctctgacttgagcgtcgattttt
gtgatgctcgtcaggggggcggagcctatggaaaaacgccagcaacgcggcctttttacggttcctggccttttgctggcctttt
gctcacatgttctttcctgcgttatcccctgattctgtggataaccgtattaccgcctttgagtgagctgataccgctcgccgca
gccgaacgaccgagcgcagcgagtcagtgagcgaggaagcggaagagcgcccaatacgcaaaccgcctctccccgcgcgttggcc
gattcattaatgcagctggcacgacaggtttcccgactggaaagcgggcagtgagcgcaacgcaattaatgtgagttagctcact
cattaggcaccccaggctttacactttatgcttccggctcgtatgttgtgtggaattgtgagcggataacaatttcacacaggaa
acagctatgaccatgattacgccaagctcgaaattaaccctcactaaagggaacaaaagctggagctccaccgcggtggcggcct
cgaggtcgagatccggtcgaccagcaaccatagtcccgcccctaactccgcccatcccgcccctaactccgcccagttccgccca
ttctccgccccatggctgactaattttttttatttatgcagaggccgaggccgcctcggcctctgagctattccagaagtagtga
ggaggcttttttggaggcctaggcttttgcaaaaagcttcgacggtatcgattggctcatgtccaacattaccgccatgttgaca
ttgattattgactagttattaatagtaatcaattacggggtcattagttcatagcccatatatggagttccgcgttacataactt
acggtaaatggcccgcctggctgaccgcccaacgacccccgcccaatgacgtcaataatgacgtatgttcccatagtaacgccaa
tagggactttccattgacgtcaatgggtggagtatttacggtaaactgcccacttggcagtacatcaagtgtatcatatgccaag
tacgccccctattgacgtcaatgacggtaaatggcccgcctggcattatgcccagtacatgaccttatgggactttcctacttgg
cagtacatctacgtattagtcatcgctattaccatggtgatgcggttttggcagtacatcaatgggcgtggatagcggtttgact
cacggggatttccaagtctccaccccattgacgtcaatgggagtttgttttggcaccaaaatcaacgggactttccaaaatgtcg
taacaactccgccccattgacgcaaatgggcggtaggcgtgtacggaattcggagtggcgagccctcagatcctgcatataagca
gctgctttttgcctgtactgggtctctctg
(nucleotide sequence encoding the CD20 CAR)
SEQ ID NO: 3
atgttgctgctggttacatctctgctgctgtgcgagctgccccatcctgcctttctgctgatccccgacatcgtgctgacacaga
gccctgccatcctgagtgcttccccaggcgagaaagtgaccatgacctgtagagccagcagcagcgtgaactacatggactggta
tcagaagaagcccggcagcagccccaagccttggatctacgccacaagcaatctggccagcggagtgcctgccagattttctggc
tctggcagcggcacaagctacagcctgacaatcagcagagtggaagccgaggatgccgccacctactactgtcagcagtggtcct
tcaatcctcctaccttcggcggaggcaccaagctggaaatcaagggctctacaagcggcggaggatctggcggtggaagtggcgg
aggcggatcttctgaagttcagctgcaacagtctggcgccgagctggttaagcctggcgcctctgtgaagatgagctgcaaggcc
agcggctacaccttcaccagctacaacatgcactgggtcaagcagacccctggacagggactcgagtggatcggagccatctatc
ccggcaatggcgacacctcctacaaccagaagttcaagggcaaagccacactgaccgccgacaagagcagcagcacagcctacat
gcagctgagcagcctgaccagcgaggacagcgccgattactactgcgccagaagcaactactacggcagctcctactggttcttc
gacgtgtggggagccggcaccacagtgacagtgtctagcgagtctaagtacggaccgccttgtcctccttgtccagctcctcctg
tggccggacctagcgtgttcctgttccccccaaagcccaaggacaccctgatgatcagccggacccccgaagtgacctgcgtggt
ggtggatgtgtcccaggaagatcccgaggtgcagttcaattggtacgtggacggcgtggaagtgcacaacgccaagaccaagccc
agagaggaacagttccagagcacctaccgggtggtgtccgtgctgacagtgctgcaccaggactggctgaacggcaaagagtaca
agtgcaaggtgtccaacaagggcctgcccagcagcatcgagaaaaccatcagcaaggccaagggccagcctcgcgagccccaggt
gtacacactgcctccaagccaggaagagatgaccaagaaccaggtgtccctgacctgtctcgtgaagggcttctaccccagcgac
attgccgtggaatgggagagcaacggccagcccgagaacaactacaagaccaccccccctgtgctggacagcgacggctcattct
tcctgtacagcagactgaccgtggacaagagccggtggcaggaaggcaacgtgttcagctgcagcgtgatgcacgaggccctgca
caaccactacacccagaagtccctgtctctgagcctgggcaagatgttctgggtgctggtggtcgtgggcggagtgctggcctgt
tacagcctgctcgtgaccgtggccttcatcatcttttgggtcaagcggggcagaaagaagctgctgtatatcttcaagcagccct
tcatgcggcccgtgcagaccacacaggaagaggacggctgctcctgccggttccccgaggaagaagaaggcggctgcgagctgag
agtgaagttcagcagaagcgccgacgcccctgcctatcagcagggccagaaccagctgtacaacgagctgaacctgggcagacgg
gaagagtacgacgtgctggataagcggagaggccgggaccctgagatgggcggcaagcctagaagaaagaacccccaggaaggcc
tgtataacgaactgcagaaagacaagatggccgaggcctacagcgagatcggaatgaagggcgagcggagaagaggcaagggcca
cgatggcctgtaccagggactgagcaccgccaccaaggatacctatgacgcactgcacatgcaggccctgccccccagactcgag
ggcggaggcgaaggcagaggatctctgctgacatgcggcgacgtggaagagaaccctggccccagaatgctgctgctcgtgacaa
gcctgctgctgtgcgagctgccccaccctgcctttctgctgatcccccggaaagtgtgcaacggcatcggcatcggagagttcaa
ggacagcctgtccatcaacgccaccaacatcaagcacttcaagaattgcaccagcatcagcggcgacctgcacatcctgccagtg
gcctttagaggcgacagcttcacccacacccccccactggatccacaggaactggatattctgaaaaccgtaaaggaaatcacag
ggtttttgctgattcaggcttggcctgaaaacaggacggacctccatgcctttgagaacctagaaatcatacgcggcaggaccaa
gcaacatggtcagttttctcttgcagtcgtcagcctgaacataacatccttgggattacgctccctcaaggagataagtgatgga
gatgtgataatttcaggaaacaaaaatttgtgctatgcaaatacaataaactggaaaaaactgtttgggacctccggtcagaaaa
ccaaaattataagcaacagaggtgaaaacagctgcaaggccacaggccaggtctgccatgccttgtgctcccccgagggctgctg
gggcccggagcccagggactgcgtctcttgccggaatgtcagccgaggcagggaatgcgtggacaagtgcaaccttctggagggt
gagccaagggagtttgtggagaactctgagtgcatacagtgccacccagagtgcctgcctcaggccatgaacatcacctgcacag
gacggggaccagacaactgtatccagtgtgcccactacattgacggcccccactgcgtcaagacctgcccggcaggagtcatggg
agaaaacaacaccctggtctggaagtacgcagacgccggccatgtgtgccacctgtgccatccaaactgcacctacggatgcact
gggccaggtcttgaaggctgtccaacgaatgggcctaagatcccgtccatcgccactgggatggtgggggccctcctcttgctgc
tggtggtggccctggggatcggcctcttcatgtga
(nucleotide sequence representing the expression vector encoding the CD20 CAR)
SEQ ID NO: 4
gttagaccagatctgagcctgggagctctctggctaactagggaacccactgcttaagcctcaataaagcttgccttgagtgctt
caagtagtgtgtgcccgtctgttgtgtgactctggtaactagagatccctcagacccttttagtcagtgtggaaaatctctagca
gtggcgcccgaacagggacttgaaagcgaaagggaaaccagaggagctctctcgacgcaggactcggcttgctgaagcgcgcacg
gcaagaggcgaggggcggcgactggtgagtacgccaaaaattttgactagcggaggctagaaggagagagatgggtgcgagagcg
tcagtattaagcgggggagaattagatcgatgggaaaaaattcggttaaggccagggggaaagaaaaaatataaattaaaacata
tagtatgggcaagcagggagctagaacgattcgcagttaatcctggcctgttagaaacatcagaaggctgtagacaaatactggg
acagctacaaccatcccttcagacaggatcagaagaacttagatcattatataatacagtagcaaccctctattgtgtgcatcaa
aggatagagataaaagacaccaaggaagctttagacaagatagaggaagagcaaaacaaaagtaagaaaaaagcacagcaagcag
cagctgacacaggacacagcaatcaggtcagccaaaattaccctatagtgcagaacatccaggggcaaatggtacatcaggccat
atcacctagaactttaaatgcatgggtaaaagtagtagaagagaaggctttcagcccagaagtgatacccatgttttcagcatta
tcagaaggagccaccccacaagatttaaacaccatgctaaacacagtggggggacatcaagcagccatgcaaatgttaaaagaga
ccatcaatgaggaagctgcaggcaaagagaagagtggtgcagagagaaaaaagagcagtgggaataggagctttgttccttgggt
tcttgggagcagcaggaagcactatgggcgcagcgtcaatgacgctgacggtacaggccagacaattattgtctggtatagtgca
gcagcagaacaatttgctgagggctattgaggcgcaacagcatctgttgcaactcacagtctggggcatcaagcagctccaggca
agaatcctggctgtggaaagatacctaaaggatcaacagctcctggggatttggggttgctctggaaaactcatttgcaccactg
ctgtgccttggatctacaaatggcagtattcatccacaattttaaaagaaaaggggggattggggggtacagtgcaggggaaaga
atagtagacataatagcaacagacatacaaactaaagaattacaaaaacaaattacaaaaattcaaaattttcgggtttattaca
gggacagcagagatccagtttggggatcaattgcatgaagaatctgcttagggttaggcgttttgcgctgcttcgcgaggatctg
cgatcgctccggtgcccgtcagtgggcagagcgcacatcgcccacagtccccgagaagttggggggaggggtcggcaattgaacc
ggtgcctagagaaggtggcgcggggtaaactgggaaagtgatgtcgtgtactggctccgcctttttcccgagggtgggggagaac
cgtatataagtgcagtagtcgccgtgaacgttctttttcgcaacgggtttgccgccagaacacagctgaagcttcgaggggctcg
catctctccttcacgcgcccgccgccctacctgaggccgccatccacgccggttgagtcgcgttctgccgcctcccgcctgtggt
gcctcctgaactgcgtccgccgtctaggtaagtttaaagctcaggtcgagaccgggcctttgtccggcgctcccttggagcctac
ctagactcagccggctctccacgctttgcctgaccctgcttgctcaactctacgtctttgtttcgttttctgttctgcgccgtta
cagatccaagctgtgaccggcgcctacggctagcgccgccaccatgttgctgctggttacatctctgctgctgtgcgagctgccc
catcctgcctttctgctgatccccgacatcgtgctgacacagagccctgccatcctgagtgcttccccaggcgagaaagtgacca
tgacctgtagagccagcagcagcgtgaactacatggactggtatcagaagaagcccggcagcagccccaagccttggatctacgc
cacaagcaatctggccagcggagtgcctgccagattttctggctctggcagcggcacaagctacagcctgacaatcagcagagtg
gaagccgaggatgccgccacctactactgtcagcagtggtccttcaatcctcctaccttcggcggaggcaccaagctggaaatca
agggctctacaagcggcggaggatctggcggtggaagtggcggaggcggatcttctgaagttcagctgcaacagtctggcgccga
gctggttaagcctggcgcctctgtgaagatgagctgcaaggccagcggctacaccttcaccagctacaacatgcactgggtcaag
cagacccctggacagggactcgagtggatcggagccatctatcccggcaatggcgacacctcctacaaccagaagttcaagggca
aagccacactgaccgccgacaagagcagcagcacagcctacatgcagctgagcagcctgaccagcgaggacagcgccgattacta
ctgcgccagaagcaactactacggcagctcctactggttcttcgacgtgtggggagccggcaccacagtgacagtgtctagcgag
tctaagtacggaccgccttgtcctccttgtccagctcctcctgtggccggacctagcgtgttcctgttccccccaaagcccaagg
acaccctgatgatcagccggacccccgaagtgacctgcgtggtggtggatgtgtcccaggaagatcccgaggtgcagttcaattg
gtacgtggacggcgtggaagtgcacaacgccaagaccaagcccagagaggaacagttccagagcacctaccgggtggtgtccgtg
ctgacagtgctgcaccaggactggctgaacggcaaagagtacaagtgcaaggtgtccaacaagggcctgcccagcagcatcgaga
aaaccatcagcaaggccaagggccagcctcgcgagccccaggtgtacacactgcctccaagccaggaagagatgaccaagaacca
ggtgtccctgacctgtctcgtgaagggcttctaccccagcgacattgccgtggaatgggagagcaacggccagcccgagaacaac
tacaagaccaccccccctgtgctggacagcgacggctcattcttcctgtacagcagactgaccgtggacaagagccggtggcagg
aaggcaacgtgttcagctgcagcgtgatgcacgaggccctgcacaaccactacacccagaagtccctgtctctgagcctgggcaa
gatgttctgggtgctggtggtcgtgggcggagtgctggcctgttacagcctgctcgtgaccgtggccttcatcatcttttgggtc
aagcggggcagaaagaagctgctgtatatcttcaagcagcccttcatgcggcccgtgcagaccacacaggaagaggacggctgct
cctgccggttccccgaggaagaagaaggcggctgcgagctgagagtgaagttcagcagaagcgccgacgcccctgcctatcagca
gggccagaaccagctgtacaacgagctgaacctgggcagacgggaagagtacgacgtgctggataagcggagaggccgggaccct
gagatgggcggcaagcctagaagaaagaacccccaggaaggcctgtataacgaactgcagaaagacaagatggccgaggcctaca
gcgagatcggaatgaagggcgagcggagaagaggcaagggccacgatggcctgtaccagggactgagcaccgccaccaaggatac
ctatgacgcactgcacatgcaggccctgccccccagactcgagggcggaggcgaaggcagaggatctctgctgacatgcggcgac
gtggaagagaaccctggccccagaatgctgctgctcgtgacaagcctgctgctgtgcgagctgccccaccctgcctttctgctga
tcccccggaaagtgtgcaacggcatcggcatcggagagttcaaggacagcctgtccatcaacgccaccaacatcaagcacttcaa
gaattgcaccagcatcagcggcgacctgcacatcctgccagtggcctttagaggcgacagcttcacccacacccccccactggat
ccacaggaactggatattctgaaaaccgtaaaggaaatcacagggtttttgctgattcaggcttggcctgaaaacaggacggacc
tccatgcctttgagaacctagaaatcatacgcggcaggaccaagcaacatggtcagttttctcttgcagtcgtcagcctgaacat
aacatccttgggattacgctccctcaaggagataagtgatggagatgtgataatttcaggaaacaaaaatttgtgctatgcaaat
acaataaactggaaaaaactgtttgggacctccggtcagaaaaccaaaattataagcaacagaggtgaaaacagctgcaaggcca
caggccaggtctgccatgccttgtgctcccccgagggctgctggggcccggagcccaggggactgcgtctcttgccggaatgtca
gccgaggcagggaatgcgtggacaagtgcaaccttctggagggtgagccaagggagtttgtggagaactctgagtgcatacagtg
ccacccagagtgcctgcctcaggccatgaacatcacctgcacaggacggggaccagacaactgtatccagtgtgcccactacatt
gacggcccccactgcgtcaagacctgcccggcaggagtcatgggagaaaacaacaccctggtctggaagtacgcagacgccggcc
atgtgtgccacctgtgccatccaaactgcacctacggatgcactgggccaggtcttgaaggctgtccaacgaatgggcctaagat
cccgtccatcgccactgggatggtgggggccctcctcttgctgctggtggtggccctggggatcggcctcttcatgtgagcggcc
gctctagacccgggctgcaggaattcgatatcaagcttatcgataatcaacctctggattacaaaatttgtgaaagattgactgg
tattcttaactatgttgctccttttacgctatgtggatacgctgctttaatgcctttgtatcatgctattgcttcccgtatggct
ttcattttctcctccttgtataaatcctggttgctgtctctttatgaggagttgtggcccgttgtcaggcaacgtggcgtggtgt
gcactgtgtttgctgacgcaacccccactggttggggcattgccaccacctgtcagctcctttccgggactttcgctttccccct
ccctattgccacggcggaactcatcgccgcctgccttgcccgctgctggacaggggctcggctgttgggcactgacaattccgtg
gtgttgtcggggaaatcatcgtcctttccttggctgctcgcctgtgttgccacctggattctgcgcgggacgtccttctgctacg
tcccttcggccctcaatccagcggaccttccttcccgcggcctgctgccggctctgcggcctcttccgcgtcttcgccttcgccc
tcagacgagtcggatctccctttgggccgcctccccgcatcgataccgtcgactagccgtacctttaagaccaatgacttacaag
gcagctgtagatcttagccatttttaaaagaaaaggggggactggaagggctaattcactcccaaagaagacaagatctgctttt
tgcctgtactgggtctctctggttagaccagatctgagcctgggagctctctggctaactagggaacccactgcttaagcctcaa
taaagcttgccttgagtgcttcaagtagtgtgtgcccgtctgttgtgtgactctggtaactagagatccctcagacccttttagt
cagtgtggaaaatctctagcagaattcgatatcaagcttatcgataccgtcgacctcgagggggggcccggtacccaattcgccc
tatagtgagtcgtattacaattcactggccgtcgttttacaacgtcgtgactgggaaaaccctggcgttacccaacttaatcgcc
ttgcagcacatccccctttcgccagctggcgtaatagcgaagaggcccgcaccgatcgcccttcccaacagttgcgcagcctgaa
tggcgaatggaaattgtaagcgttaatattttgttaaaattcgcgttaaatttttgttaaatcagctcattttttaaccaatagg
ccgaaatcggcaaaatcccttataaatcaaaagaatagaccgagatagggttgagtgttgttccagtttggaacaagagtccact
attaaagaacgtggactccaacgtcaaagggcgaaaaaccgtctatcagggcgatggcccactacgtgaaccatcaccctaatca
agttttttggggtcgaggtgccgtaaagcactaaatcggaaccctaaagggagcccccgatttagagcttgacggggaaagccgg
cgaacgtggcgagaaaggaagggaagaaagcgaaaggagcgggcgctagggcgctggcaagtgtagcggtcacgctgcgcgtaac
caccacacccgccgcgcttaatgcgccgctacagggcgcgcgtcaggtggcacttttcggggaaatgtgcgcggaacccctattt
gtttatttttctaaatacattcaaatatgtatccgctcatgagacaataaccctgataaatgcttcaataatattgaaaaaggaa
gagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttg
agagttttcgccccgaagaacgttttccaatgatgagcacttttaaagttctgctatgtggcgcggtattatcccgtattgacgc
cgggcaagagcaactcggtcgccgcatacactattctcagaatgacttggttgagtactcaccagtcacagaaaagcatcttacg
gatggcatgacagtaagagaattatgcagtgctgccataaccatgagtgataacactgcggccaacttacttctgacaacgatcg
gaggaccgaaggagctaaccgcttttttgcacacaacatgggggatcatgtaactcgccttgatcgttgggaaccggagctgaat
gaagccataccaaacgacgagcgtgacaccacgatgcctgtagcaatggcaacaacgttgcgcaaactattaactggcgaactac
ttactctagcttcccggcaacaattaatagactggatggaggcggataaagttgcaggaccacttctgcgctcggcccttccggc
tggctggtttattgctgataaatctggagccggtgagcgtgggtctcgcggtatcattgcagcactggggccagatggtaagccc
tcccgtatcgtagttatctacacgacggggagtcaggcaactatggatgaacgaaatagacagatcgctgagataggtgcctcac
tgattaagcattggtaactgtcagaccaagtttactcatatatactttagattgatttaaaacttcatttttaatttaaaaggat
ctaggtgaagatcctttttgataatctcatgaccaaaatcccttaacgtgagttttcgttccactgagcgtcagaccccgtagaa
aagatcaaaggatcttcttgagatcctttttttctgcgcgtaatctgctgcttgcaaacaaaaaaaccaccgctaccagcggtgg
tttgtttgccggatcaagagctaccaactctttttccgaaggtaactggcttcagcagagcgcagataccaaatactgttcttct
agtgtagccgtagttaggccaccacttcaagaactctgtagcaccgcctacatacctcgctctgctaatcctgttaccagtggct
gctgccagtggcgataagtcgtgtcttaccgggttggactcaagacgatagttaccggataaggcgcagcggtcgggctgaacgg
ggggttcgtgcacacagcccagcttggagcgaacgacctacaccgaactgagatacctacagcgtgagctatgagaaagcgccac
gcttcccgaagggagaaaggcggacaggtatccggtaagcggcagggtcggaacaggagagcgcacgagggagcttccaggggga
aacgcctggtatctttatagtcctgtcgggtttcgccacctctgacttgagcgtcgatttttgtgatgctcgtcaggggggcgga
gcctatggaaaaacgccagcaacgcggcctttttacggttcctggccttttgctggccttttgctcacatgttctttcctgcgtt
atcccctgattctgtggataaccgtattaccgcctttgagtgagctgataccgctcgccgcagccgaacgaccgagcgcagcgag
tcagtgagcgaggaagcggaagagcgcccaatacgcaaaccgcctctccccgcgcgttggccgattcattaatgcagctggcacg
acaggtttcccgactggaaagcgggcagtgagcgcaacgcaattaatgtgagttagctcactcattaggcaccccaggctttaca
ctttatgcttccggctcgtatgttgtgtggaattgtgagcggataacaatttcacacaggaaacagctatgaccatgattacgcc
aagctcgaaattaaccctcactaaagggaacaaaagctggagctccaccgcggtggcggcctcgaggtcgagatccggtcgacca
gcaaccatagtcccgcccctaactccgcccatcccgcccctaactccgcccagttccgcccattctccgccccatggctgactaa
ttttttttatttatgcagaggccgaggccgcctcggcctctgagctattccagaagtagtgaggaggcttttttggaggcctagg
cttttgcaaaaagcttcgacggtatcgattggctcatgtccaacattaccgccatgttgacattgattattgactagttattaat
agtaatcaattacggggtcattagttcatagcccatatatggagttccgcgttacataacttacggtaaatggcccgcctggctg
accgcccaacgacccccgcccattgacgtcaataatgacgtatgttcccatagtaacgccaatagggactttccattgacgtcaa
tgggtggagtatttacggtaaactgcccacttggcagtacatcaagtgtatcatatgccaagtacgccccctattgacgtcaatg
acggtaaatggcccgcctggcattatgcccagtacatgaccttatgggactttcctacttggcagtacatctacgtattagtcat
cgctattaccatggtgatgcggttttggcagtacatcaatgggcgtggatagcggtttgactcacggggatttccaagtctccac
cccattgacgtcaatgggagtttgttttggcaccaaaatcaacgggactttccaaaatgtcgtaacaactccgccccattgacgc
aaatgggcggtaggcgtgtacggaattcggagtggcgagccctcagatcctgcatataagcagctgctttttgcctgtactgggt
ctctg

The above-indicated nucleotide sequences of SEQ ID NO: 1 encoding the CD19 CAR and the above indicated expression vector of SEQ ID NO: 2 have been used in all of the present non-limiting experimental examples.

The above-indicated nucleotide sequences of SEQ ID NO: 3 encoding the CD20 CAR and the above indicated expression vector of SEQ ID NO: 4 have been used in all of the present non-limiting experimental examples.

### Antibodies and Flow Cytometry

Antibodies against CD19 (clone HIB19, AF647), CD20 (clone 2H7, AF647), CD38 (clone HIT2, AF488), CD138 (clone MI15, PE and unconjugated) from BioLegend (London, United Kingdom); IFN-γ (clone B27, FITC) from BD Biosciences (Heidelberg, Germany), and CD8 (clone BW135/80, VioBlue) from Miltenyi were used. For dSTORM-microscopy, an anti-CD138 antibody was conjugated to AF555 (ThermoFisher Scientific). Flow analyses were performed with a FACS Canto II (BD) machine and analyzed using Flowio software (TreeStar, Ashland, Oreg.).

### Experimental Procedures

CD19CART, CD20 CART and non-transduced control T-cells were thawed, washed and maintained overnight in T-cell medium with low-dose IL-2 (10 IU/mL). Then, 1×10⁵ T-cells were co-cultured with 2.5×10⁴ primary myeloma cells or control tumor cell lines for 4 h in the absence (for microscopy measurements) or presence of GolgiStop™ (BD). GolgiStop™-treated cells were permeabilized using the Cytofix/Cytoperm Kit (BD) and stained for intracellular IFN-γ. For flow cytometric analysis of CD19 expression, untouched primary myeloma cells were washed and stained with anti-CD38-AF488, anti-CD138-PE and anti-CD19-AF647 or AF647 isotype control according to the manufacturer's instructions and subsequently washed and analyzed. For microscopy measurements, LabTek chamber slides (Nunc™ Lab-Tek™ II Chamber Slide™ System, ThermoFisher Scientific) were coated with poly-D-lysine and primary myeloma cells (or cell lines/co-cultures) and allowed to adhere for 90 min at 37° C. Afterwards, cells were washed with PBS and stained with anti-CD38-AF488, anti-CD138-AF555 and anti-CD19-AF647, anti-CD20-AF6647 or AF647 isotype control. Cells were washed and fixed with 4% paraformaldehyde and used for dSTORM-analyses.

### Super-Resolution Imaging

For reversible photoswitching of Alexa Fluor 647, a PBS-based imaging buffer (pH 7.4) was used that contained 80 mM β-mercaptoethylamine (Sigma-Aldrich, Taufkirchen, Germany) and an oxygen scavenger system containing 3% (w/v) glucose, 4 U/mL glucose oxidase and 80 U/mL catalase. dSTORM measurements were performed as previously described^{11,12}. An Olympus IX-71 inverted microscope was used (Olympus, Hamburg, Germany) equipped with an oil-immersion objective (APON 60XOTIRF, Olympus) and a nosepiece stage (IX2-NPS, Olympus). AF647, AF555 and AF488 were excited with the appropriate laser systems (Genesis MX 639 and MX 561 from Coherent, Göttingen, Germany; iBeam smart 488 nm, Toptica, Gräfelfing, Germany). The excitation light was spectrally cleaned by appropriate bandpass filters and then focused onto the backfocal plane of the objective. To switch between different illumination modes (epi and TIRE illumination), the lens system and mirror were arranged on a linear translation stage. A polychromatic mirror (HC 410/504/582/669, Semrock, Rochester, N.Y., USA) was used to separate excitation (laser) and emitted (fluorescent) light. The fluorescence emission was collected by the same objective and transmitted by the dichroic beam splitter and several detection filters (HC 440/521/607/700, Semrock; HC 679/41, Semrock, for Alexa 647; HQ 610/75, Chroma (Bellows Falls, Vt., USA), for Alexa 555; ET 525/50, Chroma, for Alexa 488), before being projected onto two electron-multiplying CCD cameras (both iXon Ultra 897, Andor, Belfast, UK; beam splitter 635 LP, Semrock). A final pixel size of 128 nm was generated by placing additional lenses in the detection path. Excitation intensity was approximately 3.3 kW/cm². Typically, 15,000 frames were recorded with a frame rate of ^{˜}67 Hz (15 ms exposure time).

### Image Reconstruction and Data Analysis

From the recorded image stack, a table with all localizations as well as a reconstructed dSTORM image was generated using the localization software rapidSTORM 3.3²². Only CD38/CD138 double-positive cells (i.e., myeloma cells) were further analyzed for CD19 expression. Quantification of CD19 and CD20 was performed with a custom-written Mathematica (Wolfram Research, Inc., Mathematica, Version 11.2, Champaign, Ill., USA) script. The analysis routine included the following steps: fluorescent spots containing less than 800 photons per frame were discarded. Repeated localizations coming from one antibody were grouped using an alpha-shape algorithm with an alpha value of 30. It was confirmed that the overall density of detected antibodies was small enough to yield well-separated alpha-shapes. Antibody densities (CD19, CD20 or isotype) were calculated from the number of grouped localizations divided by the area of the bottom plasma membrane of each cell, as determined with a region of interest (ROI)-selector. A total of 10-80 cells per patient and condition were analyzed to obtain CD19, CD20 and isotype antibody density distributions. To distinguish between non-specific (negative subpopulation) and specific (positive subpopulation) binding of CD19 and CD20 antibodies, detected antibody density distributions were fitted to a one- or two-component log-normal distribution. Relative contributions of non-specifically and specifically bound antibodies were estimated, together with the average density (localizations μm⁻²) of specifically bound antibodies. The significance of all distribution estimates was statistically tested using an Anderson-Darling test (rejected for p-values<0.05).

### Example 1: Patient Characteristics and CD19-Expression by FC

To generate expression profiles of CD19 on primary myeloma cells by FC and dSTORM, bone marrow was obtained from 14 consecutive patients with MM that had measurable disease by histopathology. In this patient series, 4 patients had newly diagnosed myeloma, and 10 patients had been previously treated and were either in a state of partial remission (n=2) or had progressing disease (n=8) (Table S1). First, the inventors performed FC to detect CD19 on myeloma cells (FIG. 5). In two of the 14 patients (M012 and M016), the inventors found a clearly distinguishable CD19-positive myeloma cell population, comprising 30.4% and 4.9% of cells, respectively (FIG. 1A, B). In the remaining 12 patients, myeloma cells were either CD19-negative or contained only a minute population of myeloma cells (<3%) in which the signal obtained after staining for CD19 could not be discriminated from background (FIG. 1C, D; FIG. 5; Table 1).

### Example 2: dSTORM is More Sensitive Than FC in Detecting CD19 on Myeloma Cells

dSTORM was applied on the same sample of myeloma cells from the two patients who were clearly CD19-positive by FC. In both patients, the percentage of myeloma cells on which the inventors detected CD19 by dSTORM was higher compared to FC: in patient M012 68% (vs. 30.4% by FC); and in patient M016 32% (vs. 4.9% by FC) (Table 1). This discrepancy suggested that dSTORM is more sensitive than FC in detecting CD19. To test this, antibody titration experiments were performed on the human leukemia cell line NALM-6, which uniformly expresses CD19 (FIG. 6 A). The results showed that the detection limit of the dSTORM approach is 0.006±0.002 CD19 molecules/μm², which corresponds approximately to 3.1±1.3 CD19 molecules per cell for this model cell line. This value is at least 3-log-fold lower than the detection limit that has been determined for FC (FIG. 6; Table S2). Taken together, these data demonstrate that dSTORM is more sensitive than FC in detecting CD19, and able to visualize CD19 molecules on tumor cells with single-molecule resolution.

### Example 3: CD19^{low} Myeloma Cells Identified by dSTORM are Not Detected by FC

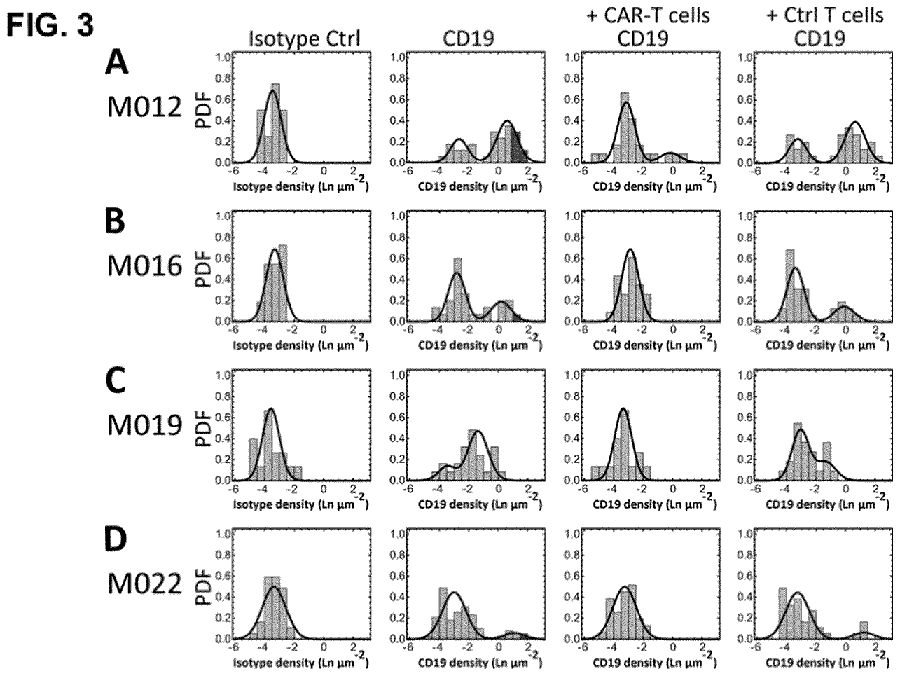

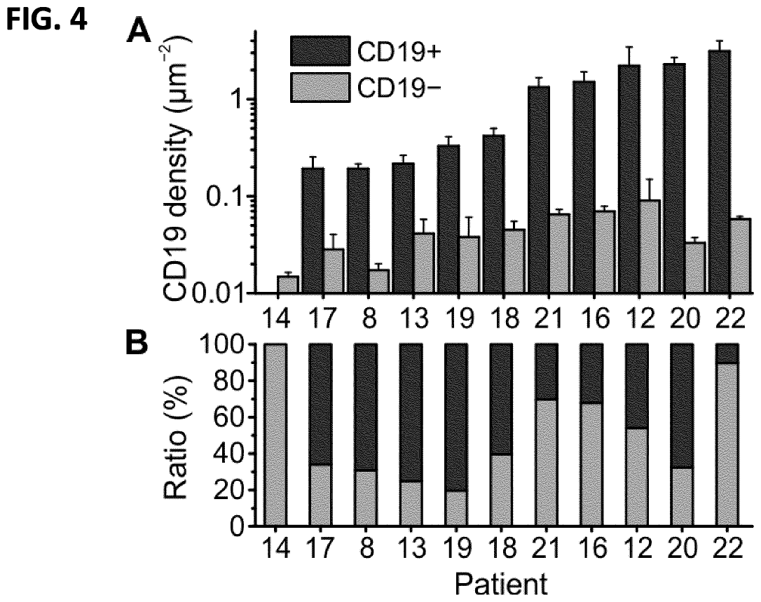

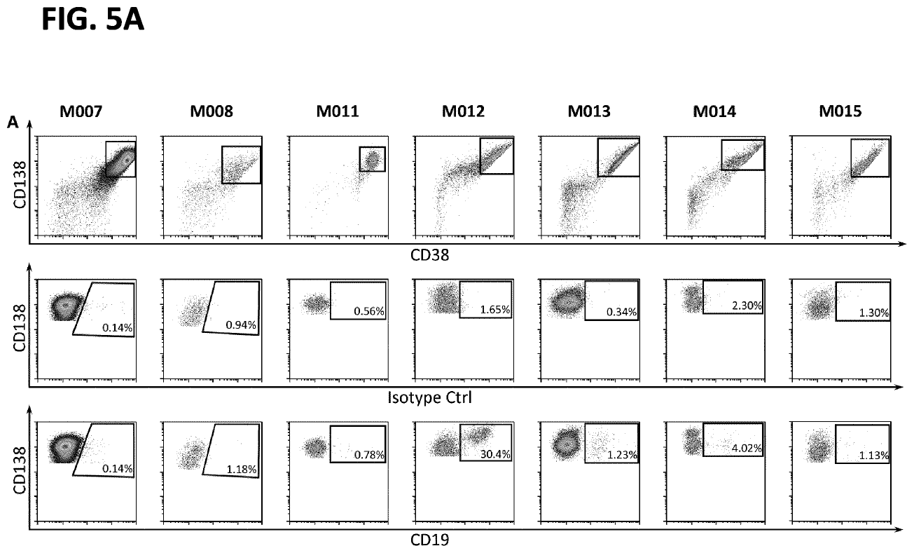

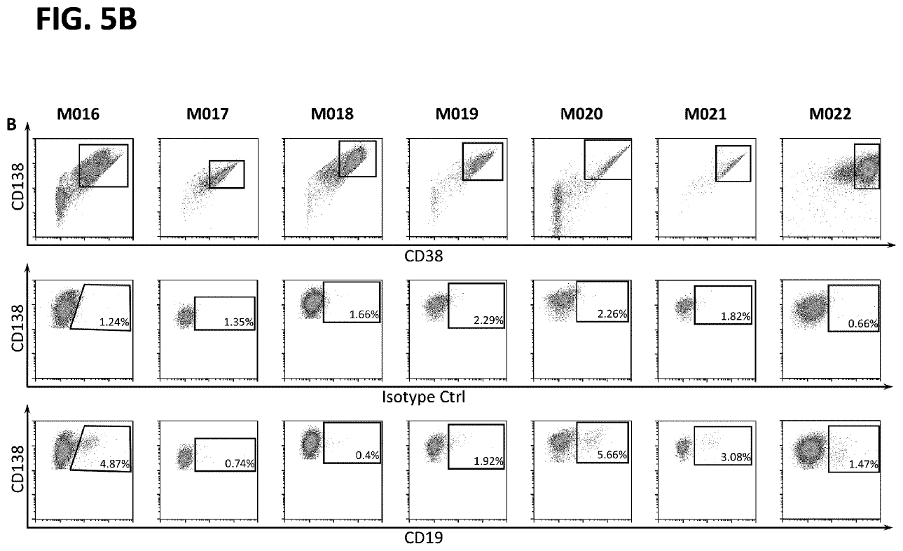

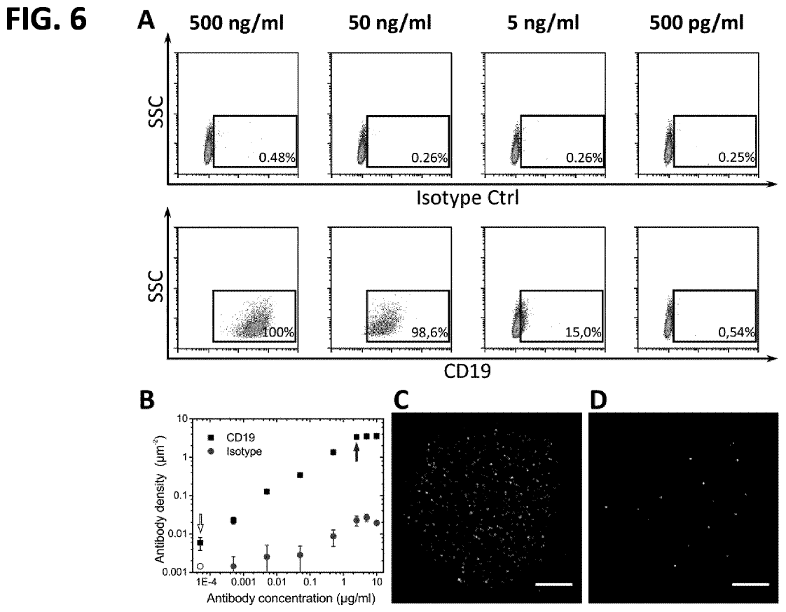

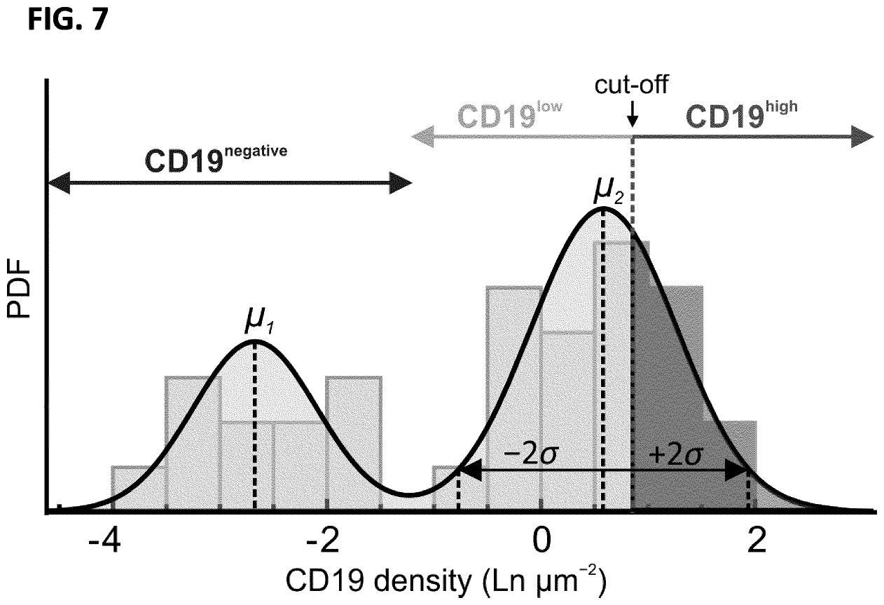

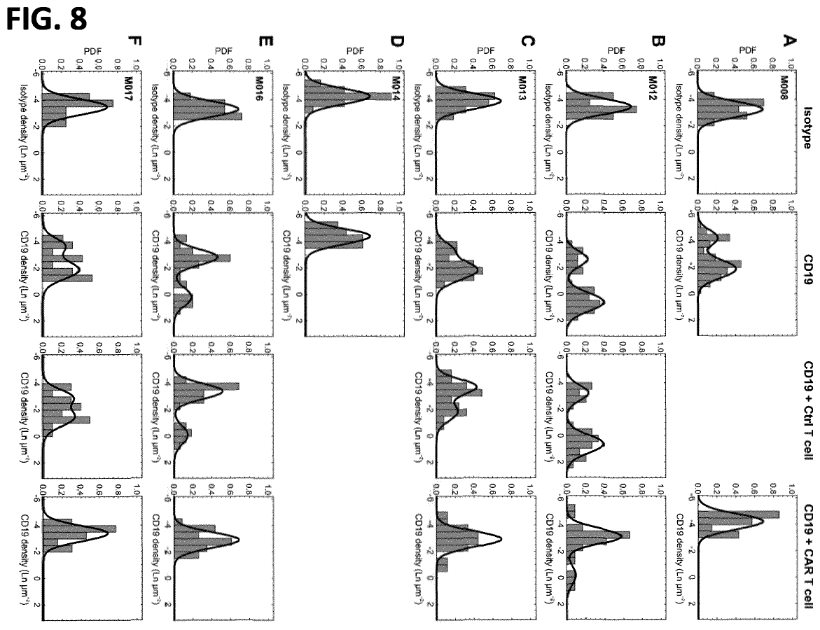

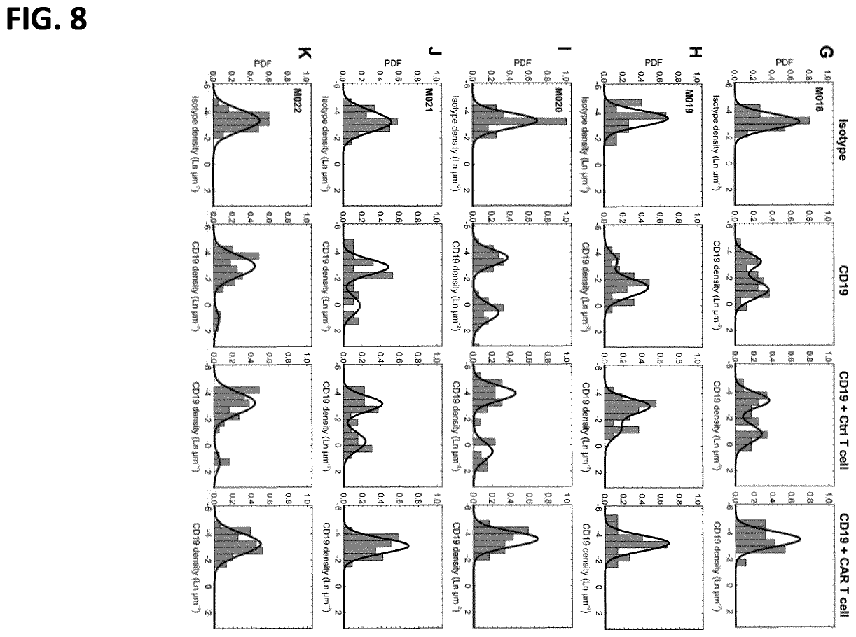

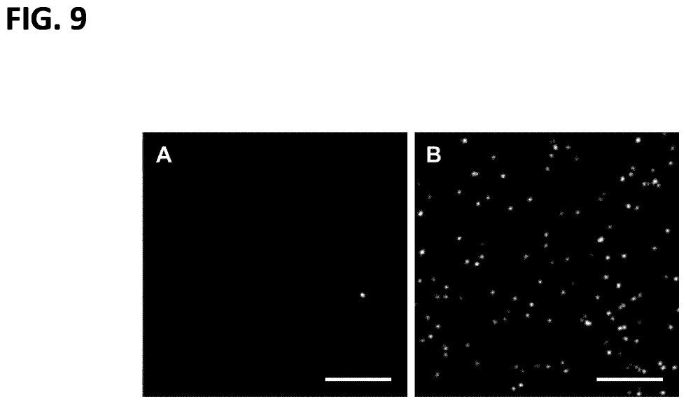

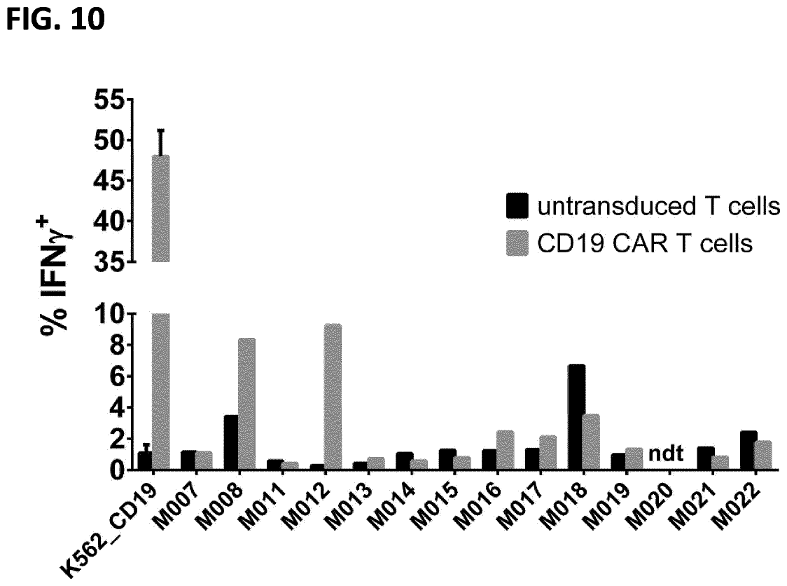

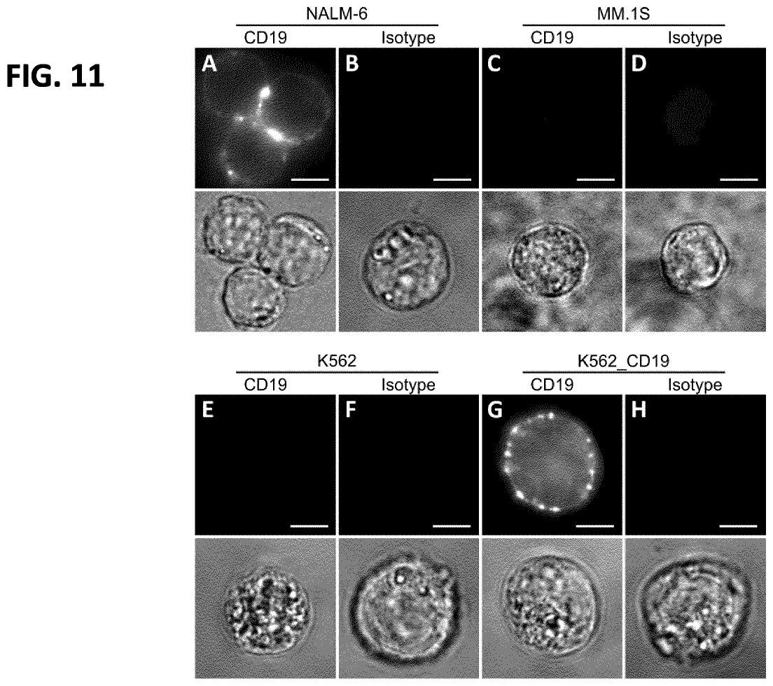

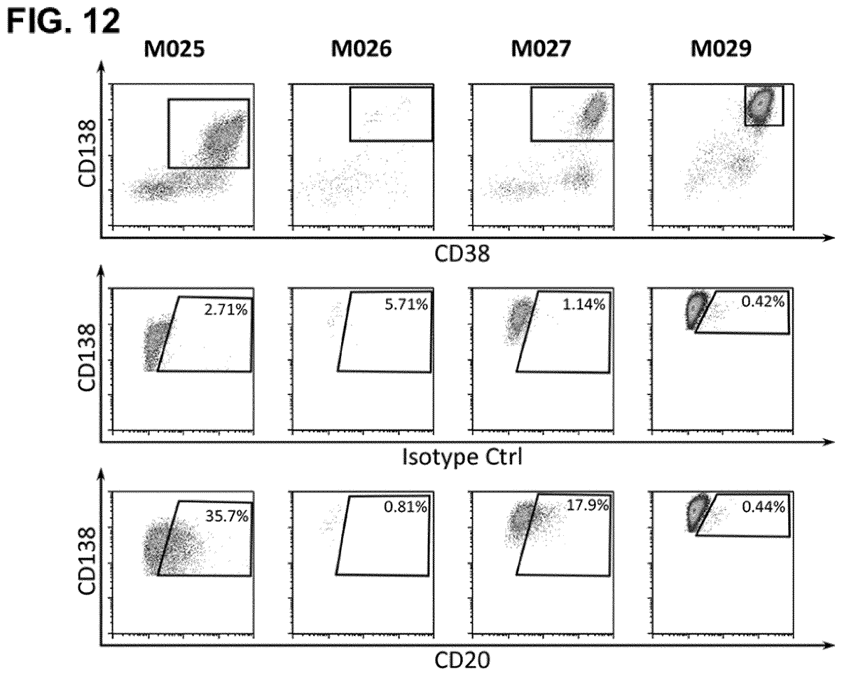

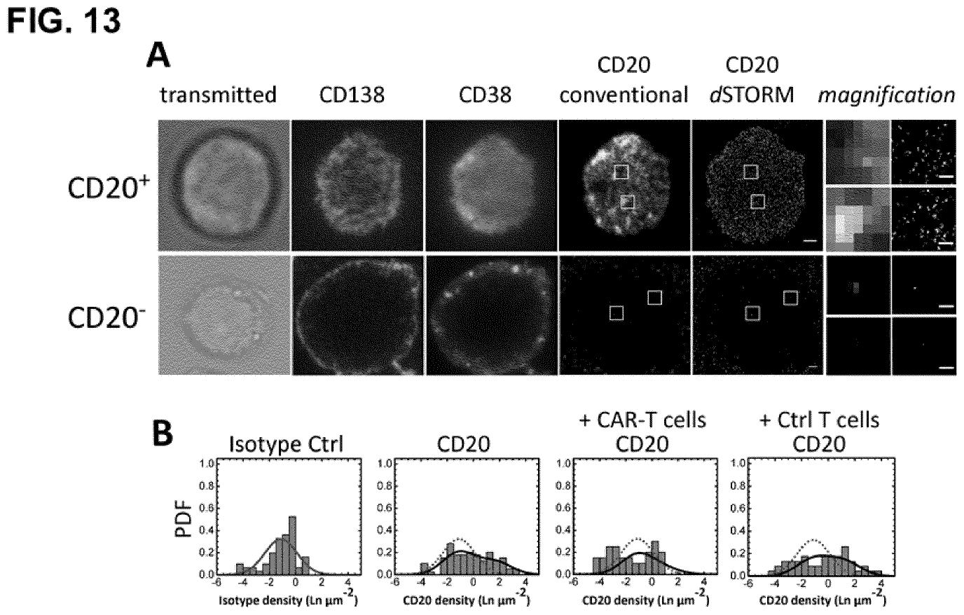

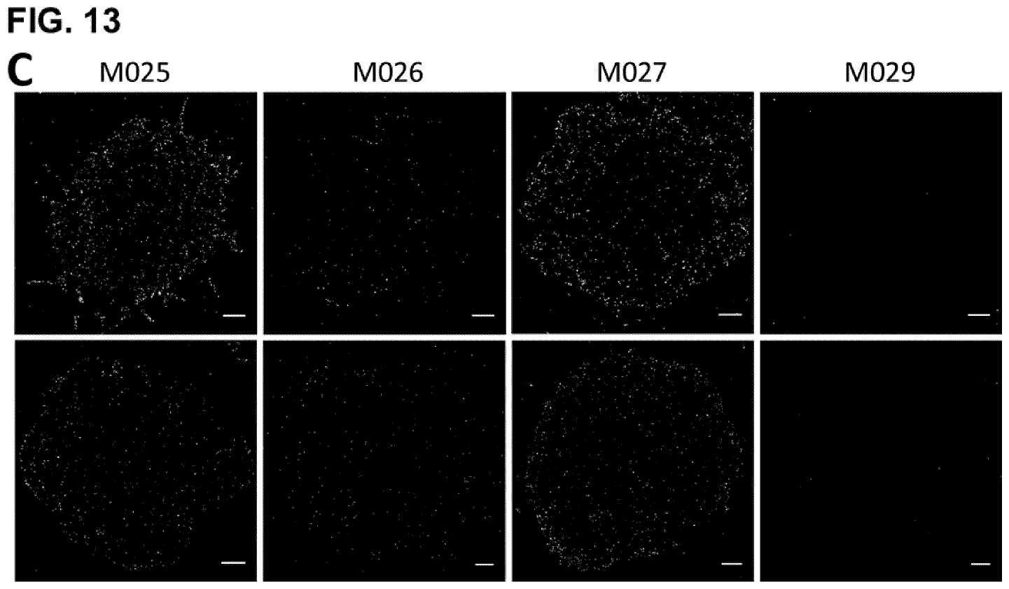

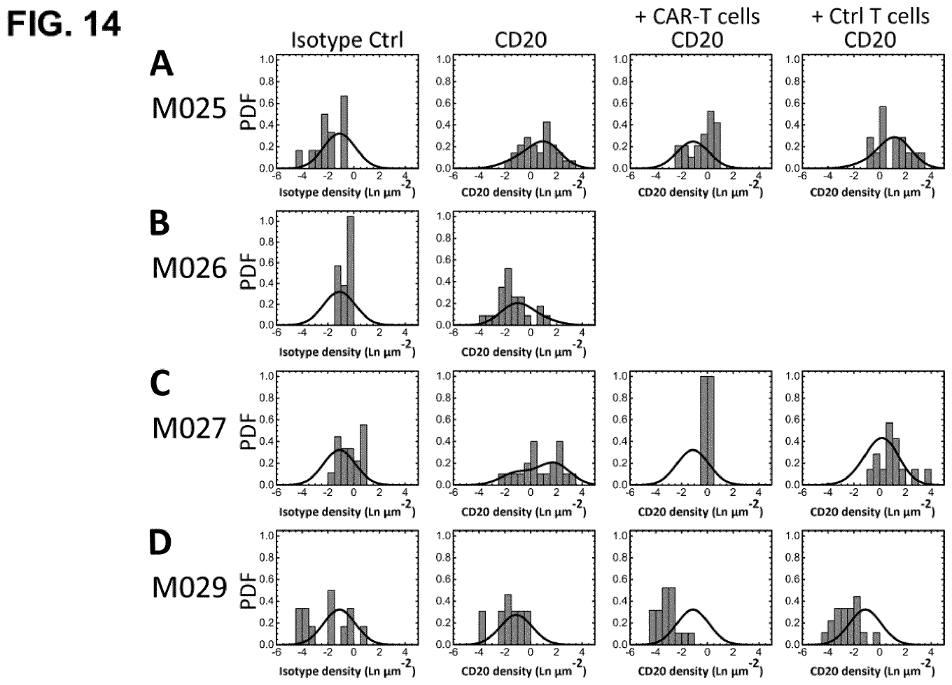

Based on the higher detection sensitivity of dSTORM compared to FC, the inventors hypothesized that, in addition to CD19^{high} myeloma cells that are detected by FC, there is an as-yet undetected population of CD19^{low} myeloma cells that is invisible to FC. To test this, flow cytometry-based cell sorting was attempted to separate CD19-positive and CD19-negative myeloma cells but it was found that the number of cells that survived this procedure was insufficient to perform subsequent dSTORM-analyses. Therefore, CD19 density plots were generated based on the dSTORM data obtained from myeloma cells of patient M012. A schematic density plot and classification is provided in FIG. 7. The plot showed a clear segregation into CD19-positive and CD19-negative myeloma cells as anticipated (FIG. 3A). The average density of CD19 on all CD19-positive myeloma cells from patient M012 was 1,200±580 molecules per cell (Table 1). The inventors reasoned that FC had only detected myeloma cells with the highest CD19-expression and quantified CD19 molecules from cells in the top 30.4% of the density plot. It was found that the average number of CD19 molecules on these CD19^{high} myeloma cells was 2,240±260 molecules per cell compared with 750±60 molecules in the remaining, CD19^{low} myeloma cells. (FIG. 3A, Table 1). The cut-off value separating CD19^{high} and CD19^{low} myeloma cells at the 30.4^{th} percentile of the density plot was 1,350 CD19 molecules per cell. The inventors obtained similar data for patient M016 (FIG. 3B). Collectively, these data show that single-molecule sensitive fluorescence imaging by dSTORM detects CD19^{low} myeloma cells that express less than 1,350 CD19 molecules per cell and are not detected by FC.

### Example 4: dSTORM Detects CD19^{low} Myeloma Cells in Patients That are Classified as CD19-Negative by FC

Next, the inventors examined CD19-expression by dSTORM on myeloma cells from the 12 patients who were classified as CD19-negative or ambiguous by FC. CD19-positive myeloma cells were detected in 8 out of these 12 patients by dSTORM (FIG. 4 A. FIG. 8, FIG. 9) and determined that they comprised between 10.3 and 80.3% of the entire myeloma cell population (mean: 55±9%, FIG. 48, Table 1). In five of these 8 patients, myeloma cells were exclusively CD19^{low}. In three of these 8 patients, a small proportion of myeloma cells with CD19^{high} expression was also detected (mean: 29±10%) (Table 1, FIG. 8). In the remaining four patients, only CD19-negative myeloma cells were detected by dSTORM at levels that were not significantly different from the background signal (mean: 17.1±2.4 molecules per cell) obtained with primary myeloma cells. Taken together, these data show that CD19 is expressed at low levels on a substantial proportion of myeloma cells in patients that are falsely classified as CD19-negative by FC.

### Example 5: CD19^{low} (and CD19^{high}) Myeloma Cells are Eliminated by CD19CART

To investigate whether CD19-expression on CD19^{high} and CD19^{low} myeloma cells is sufficient for CART recognition, the inventors treated them with CD19CART for 4 hours in vitro and then repeated the dSTORM-analysis. In all patients that contained CD19^{high} and CD19^{low} myeloma cells, it was found that CD19-expressing myeloma cells as detected by dSTORM were completely eliminated and only CD19-negative myeloma cells were present after the treatment (FIG. 3, FIG. 8). Control T-cells derived from the same donor and not equipped with the CD19-CAR did not confer any relevant reactivity against CD19^{high} and CD19^{low} myeloma cells (FIG. 3, FIG. 8). The complete elimination of CD19^{low} myeloma cells indicated that CD19CART required an antigen density of less than 1,350 CD19 molecules per target cell for being triggered. To exclude the potential that elimination of CD19^{low} myeloma cells had occurred due to bystander killing (i.e. due to cytolytic granules released from CD19CART that were triggered by CD19^{high} myeloma cells), the CD19CART treatment assay was repeated with myeloma cells that were exclusively CD19^{low}. In all patients, the inventors found, that CD19CART completely eliminated CD19^{low} myeloma cells, including CD19^{low} myeloma cells from patients M017 and M013, that expressed on average 64±8 and 93±10 CD19 molecules per cell, respectively (FIG. 8, FIG. 9). Collectively, these data demonstrate that CD19CART are capable of rapidly eliminating myeloma cells that express very low levels of CD19. Further, the data demonstrate that the antigen threshold required for triggering CD19CART is well below 100 CD19 molecules per target cell.

### Example 6: IFNγ-Secretion by CD19CART Does Not Predict the Presence of CD19^{low} Myeloma Cells

The inventors sought to determine whether intracellular staining for IFNγ production in CD19CART after co-culture with myeloma cells could be used as a simple surrogate assay to test for the presence of CD19^{low} myeloma cells instead of using single-molecule sensitive fluorescence imaging. However, the IFNγ assay worked in only two of ten patients that had been shown to contain CD19-positive myeloma cells by FC and dSTORM (FIG. 10). These data suggest that the antigen threshold required for inducing cytokine production in CD19CART is higher compared to the threshold required for triggering cytolytic activity, consistent with prior data on triggering distinct T-cell effector functions¹⁷. In summary, these data show that conventional detection and analytical methods are not sensitive enough to reveal very low level CD19 expression on myeloma cells.

### Example 7: dSTORM Detects CD19 on Primary Myeloma Cells with Single-Molecule Sensitivity

To perform dSTORM-imaging of CD19, the inventors used the B-cell acute lymphoblastic leukemia cell line NALM-6, which is uniformly positive for CD19 by flow cytometry (phenotype: CD19⁺CD38⁺CD138⁺). The K562 served as a negative control (CD19⁻, FIG. 11). The inventors first sought to establish the detection range of dSTORM-imaging and stained NALM-6 cells with serial dilutions of AF649-labeled anti-CD19 mAb (concentration (c)=5×10⁻⁵−10 μg/m; n=10-30 cells analyzed per dilution). Under saturating conditions (c≥2.5 μg/ml), the inventors detected 3.4±0.2 CD19 molecules/μm², which corresponds to 1,780±210 CD19 molecules per NALM-6 cell. With each dilution, the inventors obtained gradually lower numbers of CD19 molecules (Table S2). The detection limit of dSTORM was 0.006±0.002 CD19 molecules/μm², which corresponded to 3.1±1.3 CD19 molecules per cell (c=5×10⁻⁵ μg/ml) (FIG. 6). At all concentrations, dSTORM correctly classified all analyzed NALM-6 cells as expressing CD19 (n=176 cells). For comparison, the inventors performed parallel analyses by flow cytometry; uniform CD19 expression on NALM-6 cells was only detected when the anti-CD19 mAb was used at c≥5×10⁻² μg/ml (FIG. 6). Collectively, these data demonstrate that dSTORM-imaging is able to detect CD19 with high specificity on tumor cells at high and very low molecular densities. Furthermore, dSTORM is at least 3-log-fold more sensitive than flow cytometry in detecting very low levels of CD19 molecules on tumor cells.

### Example 8: CD20^{low} (and CD20^{high}) Myeloma Cells are Eliminated by CD20CART

To extend the applicability of the invention beyond CD19, the inventors investigated the expression of CD20, another molecule usually considered to be absent on myeloma cells in the majority of patients²⁷ on primary samples of myeloma patients using dSTORM and FC. As for CD19, CD20 was found to be infrequently expressed in 4 additional patients as judged by flow cytometry with 2/4 patients classified as uniformly CD20. In 2/4 patients, FC detected a CD20³⁰ population accounting for 33% (M025) and 16.8% (M027) of the myeloma cells. (FIG. 12).

In contrast, dSTORM revealed the existence of a CD20⁺ population in 3/4 patients accounting for 17.4-76.7% of the myeloma cells revealing the existence of a CD20^{dim} population in all patients as the size of the CD20-expressing population was found to be much higher than estimated by flow cytometry (76.7% vs. 33%, M025 and 64.7% vs. 16.8%, M027; Table 2). Calculation of the antigen density on the surface resulted in median values of 650-1,911 CD20 molecules per cell. The inventors found that, as for CD19, 4 hour cocultivation with CD2OCART led to eradication of CD20-expressing cells in 2/2 patients (FIG. 13, FIG. 14, Table S2).

TABLE S1
Patient characteristics.

| Characteristic | all patients (n = 14) | |
|---|---|---|
| Median age (range) - yr | 62.3 | (52-81) |
| Male - no. (%) | 7 | (50) |

Salmon & Durie* stage at diagnosis - no. (%)

| | | |
|---|---|---|
| I | 3 | (21) |
| II | 1 | (7) |
| IIIA | 8 | (57) |
| IIIB | 2 | (14) |

Myeloma subtype - no. (%)

| | | |
|---|---|---|
| IgG | 8 | (57) |
| IgA | 1 | (7) |
| IgD | 1 | (7) |
| LC | 4 | (29) |

Cytogenetic profile** - no. (%)

| | | |
|---|---|---|
| High-risk | 5 | (36) |
| Standard risk | 9 | (64) |
| Time from diagnosis (range) - months | 16 | (0-69) |

Remission state*** - no. (%)

| | | |
|---|---|---|
| Primary diagnosis | 4 | (29) |
| VGPR | 2 | (14) |
| Progressive disease | 8 | (57) |
| Bone marrow infiltration - % (range) | 25 | (10-99) |

Previous therapy regimens

| | | |
|---|---|---|
| Median no. (range) | 1 | (0-3) |

Previous therapies - no. (%)

| | | |
|---|---|---|
| Hematopoietic stem-cell | 9 | (64) |
| transplantation (autologous) | | |
| Lenalidomide | 6 | (43) |
| Proteasome inhibitor | 10 | (71) |

*A clinical staging system for MM based on the correlation of the measured myeloma cell mass with presenting clinical features, response to treatment, and survival.²⁸
**Cytogenetic analysis: a high-risk cytogenetic profile refers to adverse FISH including IgH translocations (t(4; 14) or t(14; 16) or t(14; 20)), 17p13 del and/or 1q21 gain.²⁹
***International Myeloma Working Group consensus criteria for response and minimal residual disease assessment in multiple myeloma.³⁰

TABLE S2
Antibody titration on NALM-6 cells

| | |
|---|---|
| Antibody | Molecules per cell |

| conc. (μg/ml) | Anti-CD19 | Isotype |
|---|---|---|
| 10 | 1880 ± 220 | 72 ± 20 |
| 5 | 1860 ± 180 | 85 ± 25 |
| 2.5 | 1780 ± 210 | 50 ± 30 |
| 0.5 | 710 ± 110 | 44 ± 28 |
| 0.05 | 182 ± 29 | 7.1 ± 6.4 |
| 5 × 10⁻³ | 67 ± 10 | 1.3 ± 1.4 |
| 5 × 10⁻⁴ | 12.0 ± 2.9 | 0.8 ± 0.6 |
| 5 × 10⁻⁵ | 3.1 ± 1.3 | 0 ± 0 |
| 0 | 0 ± 0 | 0 ± 0 |

TABLE 1
Summary of data obtained by dSTORM and flow cytometry for CD19

| | | Flow | | | | | | |
|---|---|---|---|---|---|---|---|---|
| | | Cytometry | dSTORM | | | | | IFNγ |
| | | Δ % CD19⁺ | | | | | Elimination | production |
| Patients | | (% CD19⁺ − | % | CD19 molecules/cell* | | | by | by |
| # | Identifier | % isotype⁺) | CD19⁺ | CD19⁺ (range) | CD19^{low} | CD19^{high} | CD19CART | CD19CART |
| 1 | M007 | 0 | 0 | 0 | 0 | 0 | 0 | 0 |
| | | (0.1%-0.1%) | | | | | | |
| 2 | M008 | 0 | 69.2% | 110 | 110 | 0 | + | 4.9% |
| | | (1.2%-0.9%) | | (22-340) | | | | |
| 3 | M011 | (s) | 0 | 0 | 0 | 0 | 0 | 0 |
| | | (0.8%-0.6%) | | | | | | |
| 4 | M012 | 29% | 67.6% | 1,200 | 750 | 2,240 | + | 8.9% |
| | | (30.4%-1.6%) | | (250-3,700) | | (30%) | | |
| 5 | M013 | 0.9% | 75.1% | 93 | 93 | 0 | + | 0.3% |
| | | (1.2%-0.3%) | | (19-290) | | | | |
| 6 | M014 | 1.7% | 0 | 0 | 0 | 0 | 0 | 0 |
| | | (4.0%-2.3%) | | | | | | |
| 7 | M015 | (s) | 0 | 0 | 0 | 0 | 0 | 0 |
| | | (1.1%-1.3%) | | | | | | |
| 8 | M016 | 3.7% | 32.1% | 530 | 470 | 1,850 | + | 1.2% |
| | | (4.9%-1.2%) | | (110-1,650) | | (4%) | | |
| 9 | M017 | 0 | 66.0% | 64 | 64 | 0 | + | 0.8% |
| | | (0.7%-1.4%) | | (13-200) | | | | |
| 10 | M018 | 0 | 60.4% | 270 | 270 | 0 | + | 0 |
| | | (0.4%-1.7%) | | (55-830) | | | | |
| 11 | M019 | 0 | 80.3% | 140 | 140 | 0 | + | 0.4% |
| | | (1.9%-2.3%) | | (28-420) | | | | |
| 12 | M020 | 2.4% | 46.0% | 950 | 680 | 2,090 | + | ndt |
| | | (5.7%-2.3%) | | (200-3,000) | | (19%) | | |
| 13 | M021 | 1.3% | 30.2% | 630 | 530 | 1,900 | + | 0 |
| | | (3.1%-1.8%) | | (130-2,000) | | (7%) | | |
| 14 | M022 | 0.8% | 10.3% | 1,600 | 830 | 2,500 | + | 0 |
| | | (1.5%-0.7%) | | (330-5,000) | | (47%) | | |

(s): single events
ndt: cytokine production was not assessed for patient M020
*Mean values are indicated in bold. In brackets: Calculated data ranging from small (median − 2σ) to high (median + 2σ) values (95.45% of all values lie within this range). CD19⁺ cells with more than 1,350 molecules per cell were classified as CD19^{high} and were otherwise classified as CD19^{low} (simulated data). Δ % CD19⁺: the percentage of the cells in the CD19⁺ gate for the isotype control was subtracted from the percentage of cells in the CD19⁺ gate for the respective CD19 staining.

TABLE 2
Summary of data obtained by dSTORM and flow cytometry for CD20

| | | Flow Cytometry | | | |
|---|---|---|---|---|---|
| | | Δ % CD20⁺ | dSTORM | | |
| Patients | | (% anti-CD20- | | CD20 molecules/ | Elimination by |
| # | Identifier | % isotype) | % CD20⁺ | cell* (range) | CD20CART |
| 15 | M025 | 33 | 76.7 | 650 | |
| | | (35.7-2.7) | | (55-7, 724) | |
| 16 | M026 | 0 | 17.4 | 1,911 | ndt |
| | | (0-5.7) | | (160-22, 719) | |
| 17 | M027 | 16.8 | 64.7 | 1,770 | + |
| | | (17.9-1.1) | | (149-21, 045) | |
| 18 | M029 | 0 | 0 | 0 | 0 |
| | | (0.42-0.44) | | | |

ndt: cytoxicity was not assessed for patient M026
*Mean values are indicated in bold. In brackets: Calculated data ranging from small (median − 2σ) to high (median + 2σ) values (95.45% of all values lie within this range).
Δ % CD20⁺: the percentage of the cells in the CD20⁺ gate for the isotype control was subtracted from the percentage of cells in the CD20⁺ gate for the respective CD20 staining.

### INDUSTRIAL APPLICABILITY

The immune cells for the uses according to the invention, as well as materials used for the methods of the invention, may be industrially manufactured and sold as products for the claimed methods and uses (e.g. for treating a cancer as defined herein), in accordance with known standards for the manufacture of pharmaceutical and diagnostic products. Accordingly, the present invention is industrially applicable.

### REFERENCES

1. Kumar S K, Dispenzieri A, Lacy M Q, et al. Continued improvement in survival in multiple myeloma: changes in early mortality and outcomes in older patients. Leukemia 2014;28:1122-8.

2. Garfall A L, Maus M V, Hwang W T, et al. Chimeric Antigen Receptor T Cells against CD19 for Multiple Myeloma. The New England journal of medicine 2015;373:1040-7.

3. Turtle C J , Hanafi L A, Berger C, et al. Immunotherapy of non-Hodgkin's lymphoma with a defined ratio of CD8+ and CD4+ CD19-specific chimeric antigen receptor-modified T cells. Science translational medicine 2016;8:355ra116.

4. Turtle C I, Hanafi L A, Berger C, et al. CD19 CAR-T cells of defined CD4+:CD8+ composition in adult B cell ALL patients. J Clin Invest 2016;126:2123-38.

5. Bach P B, Giralt S A, Saltz L B. FDA Approval of Tisagenlecleucel: Promise and Complexities of a $475000 Cancer Drug. JAMA: the journal of the American Medical Association 2017.

6. Roberts L I, Better M, Bot A, Roberts M R, Ribas A. Axicabtagene ciloleucel, a first-in-class CAR T cell therapy for aggressive NHL. Leukemia & lymphoma 2017:1-12.

7. Uckun F M, Qazi S, Dibirdik I, Myers D E. Rational design of an immunoconjugate for selective knock-down of leukemia-specific E2A-PBX1 fusion gene expression in human Pre-B leukemia. Integr Biol (Camb) 2013;5:122-32.

8. Tembhare P R, Yuan C M, Venzon D, et al. Flow cytometric differentiation of abnormal and normal plasma cells in the bone marrow in patients with multiple myeloma and its precursor diseases. Leukemia research 2014;38:371-6.

9. Ehmann N, van de Linde 5, Alon A, et al. Quantitative super-resolution imaging of Bruchpilot distinguishes active zone states. Nat Commun 2014;5:4650.

10. Letschert S, Göhler A, Franke C, et al. Super-resolution imaging of plasma membrane glycans. Angewandte Chemie 2014;53:10921-4.

11. Heilemann M, van de Linde S, Schüttpelz M, et al. Subdiffraction-resolution fluorescence imaging with conventional fluorescent probes. Angewandte Chemie 2008;47:6172-6.

12. van de Linde S, Löschberger A, Klein T, et al. Direct stochastic optical reconstruction microscopy with standard fluorescent probes. Nature protocols 2011;6:991-1009.

13. Hajek R, Okubote S A, Svachova H. Myeloma stem cell concepts, heterogeneity and plasticity of multiple myeloma. British journal of haematology 2013;163:551-64.

14. Paiva B, Puig N, Cedena M T, et al. Differentiation stage of myeloma plasma cells: biological and clinical significance. Leukemia 2016.

15. Zola H. High-sensitivity immunofluorescence/flaw cytometry: detection of cytokine receptors and other low-abundance membrane molecules2004.

16. Truneh A, Machy P. Detection of very low receptor numbers on cells by flow cytometry using a sensitive staining method. Cytometry 1987;8:562-7.

17. Stone J D, Aggen D H, Schietinger A, Schreiber H, Kranz D M. A sensitivity scale for targeting T cells with chimeric antigen receptors (CARs) and bispecific T-cell Engagers (BiTEs). Oncoimmunology 2012;1:863-73.

18. Watanabe K, Terakura S, Martens A C, et al. Target antigen density governs the efficacy of anti-CD20-CD28-CD3 zeta chimeric antigen receptor-modified effector CD8+ T cells. Journal of immunology 2015;194:911-20.

19. Ali S A, Shi V, Maric I, et al. T cells expressing an anti-B-cell maturation antigen chimeric antigen receptor cause remissions of multiple myeloma. Blood 2016;128:1688-700.

20. Ruella M, Maus M V. Catch me if you can: Leukemia Escape after CD19-Directed T Cell Immunotherapies. Comput Struct Biotechnol J 2016;14:357-62.

21. Sommermeyer D, Hudecek M, Kosasih P L, et al. Chimeric antigen receptor-modified T cells derived from defined CD8(+) and CD4(+) subsets confer superior antitumor reactivity in vivo. Leukemia 2016;30:492-500.

22. Wolter S, Löschberger A, Holm T, et al. rapidSTORM: accurate, fast open-source software for localization microscopy. Nat Methods 2012;9:1040-1.

23. Wang X, Chang W C, Wong C W, et al. A transgene-encoded cell surface polypeptide for selection, in vivo tracking, and ablation of engineered cells. Blood 2011;118:1255-63.

24. Terakura 5, Yamamoto T N, Gardner R A, Turtle C J, Jensen M C, Riddell S R. Generation of CD19-chimeric antigen receptor modified CD8+ T cells derived from virus-specific central memory T cells. Blood 2012;119:72-82.

25. Hudecek M, Lupo-Stanghellini M T, Kosasih P L, et al. Receptor affinity and extracellular domain modifications affect tumor recognition by ROR1-specific chimeric antigen receptor T cells. Clinical cancer research : an official journal of the American Association for Cancer Research 2013;19:3153-64.

26. Hudecek M, Sommermeyer D, Kosasih P L, et al. The nonsignaling extracellular spacer domain of chimeric antigen receptors is decisive for in vivo antitumor activity. Cancer immunology research 2015;3:125-35.

27. Huang B, Li I, Liu J, Gu J. CD20-positive multiple myeloma: can conventional chemotherapy still be used to achieve ideal outcome for these patients? Leukemia & lymphoma 2016;57:335-40.

28. Durie B G, Salmon S E. A clinical staging system for multiple myeloma. Correlation of measured myeloma cell mass with presenting clinical features, response to treatment, and survival. Cancer 1975;36:842-54.

29. Chng W J, Dispenzieri A, Chim C S, et al. IMWG consensus on risk stratification in multiple myeloma. Leukemia 2014;28:269-77.

30. Kumar S, Paiva B, Anderson K C, et al. International Myeloma Working Group consensus criteria for response and minimal residual disease assessment in multiple myeloma. Lancet Oncol 2016;17:e328-e46.

1. A method, comprising steps of:
(A) Analyzing a cancer cell-containing sample from a cancer patient to obtain information about a cell surface antigen of the cancer cell; and
(B) Classifying said cancer cell-containing sample based on the information obtained in step (A).

2. The method of claim 1, wherein said cancer is a hematologic or solid tumor.

3. The method of claim 1 or 2, wherein said cancer is leukemia, lymphoma, or myeloma, preferably wherein said cancer is multiple myeloma.

4. The method of any one of claims 1 to 3, wherein step (A) comprises analyzing the cancer cell-containing sample using super-resolution microscopy.

5. The method of any one of claims 1 to 4, wherein step (A) comprises determining the number of molecules of said cell surface antigen on said cancer cell.

6. The method of claim 4 or 5, wherein said super-resolution microscopy is single-molecule localization microscopy.

7. The method of claims 4 to 6, wherein said super-resolution microscopy is dSTORM, STORM, PALM, or FPALM.

8. The method of claim 7, wherein said super-resolution microscopy is dSTORM.

9. The method of any one of claims 1 to 8, wherein in step (A) of the method, said cancer cell-containing sample is analyzed as to whether it comprises a fraction of cancer cells which express a cell surface antigen.

10. The method of any one of claims 6 to 9, wherein the cell surface antigen is the antigen according to claim 5.

11. The method of any one of claims 5 to 10, wherein the cell surface antigen is a cancer antigen.

12. The method of any one of claims 5 to 11, wherein said cell surface antigen is not detectable by flow cytometry.

13. The method of any one of claims 5 to 12, wherein said cell surface antigen is detectable by super-resolution microscopy.

14. The method of any one of claims 5 to 13, wherein said cell surface antigen is detectable by single-molecule localization microscopy.

15. The method of any one of claims 5 to 14, wherein said cell surface antigen is detectable by dSTORM, STORM, PALM, or FPALM.

16. The method of claim 15, wherein said cell surface antigen is detectable by dSTORM.

17. The method of any one of claims 5 to 16, wherein in step (A) of the method, said cancer cell-containing sample is analyzed as to whether it comprises a fraction of cancer cells which express said cell surface antigen at a number of more than 4 cell surface antigen molecules per cell.

18. The method of any one of claims 5 to 16, wherein in step (A) of the method, said cancer cell-containing sample is analyzed as to whether it comprises a fraction of cancer cells which express said cell surface antigen at a number of more than 8 cell surface antigen molecules per cell.

19. The method of any one of claims 5 to 16, wherein in step (A) of the method, said cancer cell-containing sample is analyzed as to whether it comprises a fraction of cancer cells which express said cell surface antigen at a number of more than 16 cell surface antigen molecules per cell.

20. The method of any one of claims 5 to 16, wherein in step (A) of the method, said cancer cell-containing sample is analyzed as to whether it comprises a fraction of cancer cells which express said cell surface antigen at a number of more than 32 cell surface antigen molecules per cell.

21. The method of any one of claims 5 to 16, wherein in step (A) of the method, said cancer cell-containing sample is analyzed as to whether it comprises a fraction of cancer cells which express said cell surface antigen at a number of more than 64 cell surface antigen molecules per cell.

22. The method of any one of claims 5 to 16, wherein in step (A) of the method, said cancer cell-containing sample is analyzed as to whether it comprises a fraction of cancer cells which express said cell surface antigen at a number of more than 100 cell surface antigen molecules per cell.

23. The method of any one of claims 5 to 16, wherein in step (A) of the method, said cancer cell-containing sample is analyzed as to whether it comprises a fraction of cancer cells which express said cell surface antigen at a number of more than 200 cell surface antigen molecules per cell.

24. The method of any one of claims 5 to 16, wherein in step (A) of the method, said cancer cell-containing sample is analyzed as to whether it comprises a fraction of cancer cells which express said cell surface antigen at a number of more than 300 cell surface antigen molecules per cell.

25. The method of any one of claims 5 to 24, wherein in step (A) of the method, said cancer cell-containing sample is analyzed as to whether it comprises a fraction of cancer cells which express said cell surface antigen at a number of no more than 10,000 cell surface antigen molecules per cell.

26. The method of any one of claims 5 to 24, wherein in step (A) of the method, said cancer cell-containing sample is analyzed as to whether it comprises a fraction of cancer cells which express said cell surface antigen at a number of no more than 5,000 cell surface antigen molecules per cell.

27. The method of any one of claims 5 to 24, wherein in step (A) of the method, said cancer cell-containing sample is analyzed as to whether it comprises a fraction of cancer cells which express said cell surface antigen at a number of no more than 2,500 cell surface antigen molecules per cell.

28. The method of any one of claims 5 to 24, wherein in step (A) of the method, said cancer cell-containing sample is analyzed as to whether it comprises a fraction of cancer cells which express said cell surface antigen at a number of no more than 1,500 cell surface antigen molecules per cell.

29. The method of any one of claims 5 to 24, wherein in step (A) of the method, said cancer cell-containing sample is analyzed as to whether it comprises a fraction of cancer cells which express said cell surface antigen at a number of no more than 1,350 cell surface antigen molecules per cell.

30. The method of any one of claims 5 to 24, wherein in step (A) of the method, said cancer cell-containing sample is analyzed as to whether it comprises a fraction of cancer cells which express said cell surface antigen at a number of no more than 1,300 cell surface antigen molecules per cell.

31. The method of any one of claims 5 to 24, wherein in step (A) of the method, said cancer cell-containing sample is analyzed as to whether it comprises a fraction of cancer cells which express said cell surface antigen at a number of no more than 1,000 cell surface antigen molecules per cell.

32. The method of any one of claims 5 to 24, wherein in step (A) of the method, said cancer cell-containing sample is analyzed as to whether it comprises a fraction of cancer cells which express said cell surface antigen at a number of no more than 800 cell surface antigen molecules per cell.

33. The method of any one of claims 5 to 24, wherein in step (A) of the method, said cancer cell-containing sample is analyzed as to whether it comprises a fraction of cancer cells which express said cell surface antigen at a number of no more than 500 cell surface antigen molecules per cell.

34. The method of any one of claims 5 to 24, wherein in step (A) of the method, said cancer cell-containing sample is analyzed as to whether it comprises a fraction of cancer cells which express said cell surface antigen at a number of no more than 400 cell surface antigen molecules per cell.

35. The method of any one of claims 5 to 34, wherein said number of molecules of said cell surface antigen per cell is determined by microscopy.

36. The method of any one of claims 5 to 35, wherein said number of molecules of said cell surface antigen per cell is determined by super-resolution microscopy.

37. The method of any one of claims 5 to 36, wherein said number of molecules of said cell surface antigen per cell is determined by single-molecule localization microscopy.

38. The method of any one of claims 35 to 37, wherein said microscopy is dSTORM, STORM, PALM, or FPALM.

39. The method of any one of claims 35 to 38, wherein said microscopy is dSTORM.

40. The method of any one of claims 1 to 39, wherein said cell surface antigen is selected from the group consisting of CD19, CD20, CD22, CD27, CD30, CD33, CD38, CD44v6, CD52, CD64, CD70, CD72, CD123, CD135, CD138, CD220, CD269, CD319, ROR1, ROR2, SLAMF7, BCMA, αvβ3-Integrin, α4β1-Integrin, EpCAM-1, MUC-1, MUC-16, L1-CAM, c-kit, NKG2D, NKG2D-Ligand, PD-L1, PD-L2, Lewis-Y, CAIX, CEA, c-MET, EGFR, EGFRvIII, ErbB2, Her2, FAP, FR-a, EphA2, GD2, GD3, GPC3, IL-13Ra, Mesothelin, PSMA, PSCA, and VEGFR, preferably CD19 and/or CD20.

41. The method of any one of claims 5 to 40, wherein said cell surface antigen is CD19.

42. The method of any one of claims 5 to 41, wherein said cell surface antigen is CD20.

43. The method of any one of claims 4 to 42, wherein step (A) comprises sub-steps of:
(A-I) Labeling said cell surface antigen on said cancer cells;
(A-II) Detecting said labelled cell surface antigen on said cancer cells by super-resolution microscopy; and
(A-III) Counting the number of labelled cell surface antigen molecules per cancer cell.

44. The method of any one of claims 5 to 43, wherein said cell surface antigen is labelled in step (A) and step (A-I), respectively, by immunostaining.

45. The method of any one of claims 1 to 44, wherein step (B) further comprises steps of:
(B-I) Classifying said cancer cell containing sample as positive for said cell surface antigen if the number of cell surface antigen molecules per cell obtained in step (A-III) is above a minimum threshold; and/or
(B-II) Classifying said cancer cell containing sample as negative for said cell surface antigen if the number of cell surface antigen molecules per cell obtained in step (A-III) is below a minimum threshold.

46. The method of claim 45, wherein said minimum threshold is in the range of 4 to 300.

47. The method of claim 45 or 46, wherein said minimum threshold is 4.

48. The method of claim 45 or 46, wherein said minimum threshold is 8.

49. The method of claim 45 or 46, wherein said minimum threshold is 16.

50. The method of claim 45 or 46, wherein said minimum threshold is 32.

51. The method of claim 45 or 46, wherein said minimum threshold is 64.

52. The method of claim 45 or 46, wherein said minimum threshold is 100.

53. The method of claim 45 or 46, wherein said minimum threshold is 200.

54. The method of claim 45 or 46, wherein said minimum threshold is 300.

55. The method of any one of claims 1 to 54, wherein based on the classification of said cancer cell-containing sample in step (B), a prediction on the eligibility of said patient for cancer therapy is made.

56. The method of claim 55, wherein said patient is predicted to be eligible for cancer therapy if said classification of said cancer cell containing sample in step (B) for said cell surface antigen is positive.

57. The method of any one of claims 1 to 56, wherein the method is a method for selecting a target antigen for cancer therapy.

58. The method of any one of claims 1 to 57, wherein the method is a method for selecting a patient for cancer therapy.

59. The method of claim 58, wherein said cancer therapy is cancer immunotherapy against said cell surface antigen.

60. The method of claim 59, wherein said cancer immunotherapy is a targeted cancer immunotherapy against said cell surface antigen.

61. The method of claim 60, wherein said targeted cancer immunotherapy is a cell-based targeted cancer immunotherapy against said cell surface antigen.

62. The method of claim 61, wherein said cell-based targeted cancer immunotherapy is an immunotherapy against said cell surface antigen with chimeric antigen receptor (CAR)-engineered T-cells.

63. The method of any one of claims 59 to 62, wherein said immunotherapy is an immunotherapy targeting a cell surface antigen selected from the group consisting of CD19, CD20, CD22, CD27, CD30, CD33, CD38, CD44v6, CD52, CD64, CD70, CD72, CD123, CD135, CD138, CD220, CD269, CD319, ROR1, ROR2, SLAMF7, BCMA, αvβ3-Integrin, α4β1-Integrin, EpCAM-1, MUC-1, MUC-16, L1-CAM, c-kit, NKG2D, NKG2D-Ligand, PD-L1, PD-L2, Lewis-Y, CAIX, CEA, c-MET, EGFR, EGFRvIII, ErbB2, Her2, FAP, FR-a, EphA2, GD2, GD3, GPC3, IL-13Ra, Mesothelin, PSMA, PSCA, VEGFR, preferably wherein said immunotherapy is an immunotherapy targeting CD19 and/or CD20.

64. The method of claim 63, wherein said immunotherapy is an immunotherapy targeting CD19 and/or CD20.

65. The method of claim 63, wherein said immunotherapy is an immunotherapy targeting CD19.

66. The method of claim 63, wherein said immunotherapy is an immunotherapy targeting CD20.

67. The method of any one of claims 1 to 66, wherein all the steps are of the method are carried out in vitro.

68. The method of any one of claims 1 to 67, wherein the method does not comprise treatment of the human or animal body by surgery or therapy.

69. The method of any one of claims 1 to 68, wherein the method is not a diagnostic method practiced on the human or animal body.

70. The method of any one of claims 1 to 69, wherein said cancer cell-containing sample is a bone marrow aspirate.

71. The method of any one of claims 1 to 70, wherein said cancer cell-containing sample comprises primary myeloma cells and the patient is a myeloma patient.

72. The method of any one of claims 1 to 71, wherein said cancer cell-containing sample comprises primary myeloma cells expressing CD138 and the patient is a myeloma patient.

73. The method of any one of claims 1 to 72, wherein said cancer cell containing sample is obtainable by positive selection of primary myeloblasts from bone marrow aspirate for CD138.

74. The method of claim 73, wherein said selection is selection using magnetic beads.

75. An immune cell capable of targeting a cell surface antigen of a cell of a cancer, for use in a method for the treatment of said cancer in a patient, wherein in the method, the immune cell is to be administered to the patient.

76. The immune cell of claim 75 for use of claim 75, wherein said cancer is myeloma.

77. The immune cell of claim 75 or 76 for use of claim 75 or 76, wherein said cancer contains a fraction of cells positive for said cell surface antigen as determined according to any one of claims 5 to 74.

78. The immune cell of any one of claims 75 to 77 for the use of any one of claims 75 to 77, wherein the method comprises cancer immunotherapy.

79. The immune cell of claim 78 for the use of claim 78, wherein said cancer immunotherapy is a targeted cancer immunotherapy.

80. The immune cell of claim 79 for the use of claim 79, wherein said targeted cancer immunotherapy is a cell-based targeted cancer immunotherapy.

81. The immune cell of claim 79 or 80 for the use of claim 79 or 80, wherein said targeted cancer immunotherapy is a targeted cancer immunotherapy targeting a cell surface antigen as defined in any one of claims 63 to 66.

82. The immune cell of claim 81 for the use of claim 81, wherein the immune cell is capable of binding to said cell surface antigen.

83. The immune cell of any one of claims 75 to 82 for the use of claims 75 to 82, wherein the immune cell is capable of binding to CD19 and/or CD20.

84. The immune cell of any one of claims 75 to 83 for the use of claims 75 to 83, wherein the immune cell is capable of binding to CD20.

85. The immune cell of claim 84 for the use of claim 84, wherein the immune cell is capable of binding to the extracellular domain of CD20.

86. The immune cell of any one of claims 75 to 85 for the use of claims 75 to 85, wherein the immune cell is capable of binding to CD19.

87. The immune cell of claim 86 for the use of claim 86, wherein the immune cell is capable of binding to the extracellular domain of CD19.

88. The immune cell of any one of claims 75 to 87 for use of claims 75 to 87, wherein the cell is a cell expressing a chimeric antigen receptor.

89. The immune cell of claim 88 for use of claim 88, wherein the chimeric antigen receptor is capable of binding to said cell surface antigen.

90. The immune cell of claim 88 or 89 for use of claim 88 or 89, wherein the chimeric antigen receptor is capable of binding to CD19 and/or CD20.

91. The immune cell of any one of claims 88 to 90 for use of claims 88 to 90, wherein the chimeric antigen receptor is capable of binding to CD20.

92. The immune cell of any one of claims 88 to 91 for use of claims 88 to 91, wherein the chimeric antigen receptor is capable of binding to CD19.

93. The immune cell of any one of claims 75 to 92 for use of any one of claims 75 to 92, wherein the cell is a cell selected from the group of T cells, NK cells, and B cells.

94. The immune cell of any one of claims 75 to 93 for use of any one of claims 75 to 93, wherein the cell is a T cell.

95. The immune cell of any one of claims 75 to 94 for the use of any one of claims 75 to 94, wherein said cell-based targeted cancer immunotherapy is an immunotherapy with chimeric antigen receptor (CAR)-engineered T-cells.

96. The immune cell of any one of claims 75 to 95 for the use of any one of claims 75 to 95, wherein said patient is a patient eligible for said treatment as predictable by the method of any one of claims 55 to 74.

97. The immune cell of any one of claims 75 to 96 for the use of any one of claims 75 to 96, wherein the cancer is negative for expression of said cell surface antigen as determined by flow cytometry.

98. The immune cell of claim 97 for the use of claim 97, wherein the cancer is positive for expression of said cell surface antigen as determined by super-resolution microscopy.

99. The immune cell of claim 98 for the use of claim 98, wherein the cancer is positive for expression of said cell surface antigen as determined by single-molecule localization microscopy.

100. The immune cell of claim 98 or 99 for the use of claim 98 or 99, wherein the cancer is positive for expression of said cell surface antigen as determined by dSTORM, STORM, PALM, or FPALM.

101. The immune cell of claim 100 for the use of claim 100, wherein the cancer is positive for expression of said cell surface antigen as determined by dSTORM.

102. The immune cell of any one of claims 75 to 101 for the use of any one of claims 75 to 101, wherein a fraction of the cancer cells expresses said cell surface antigen at a number of at least 4 cell surface antigen molecules per cell.

103. The immune cell of any one of claims 75 to 101 for the use of any one of claims 75 to 101, wherein a fraction of the cancer cells expresses said cell surface antigen at a number of at least 8 cell surface antigen molecules per cell.

104. The immune cell of any one of claims 75 to 101 for the use of any one of claims 75 to 101, wherein a fraction of the cancer cells expresses said cell surface antigen at a number of at least 16 cell surface antigen molecules per cell.

105. The immune cell of any one of claims 75 to 101 for the use of any one of claims 75 to 101, wherein a fraction of the cancer cells expresses said cell surface antigen at a number of at least 32 cell surface antigen molecules per cell.

106. The immune cell of any one of claims 75 to 101 for the use of any one of claims 75 to 101, wherein a fraction of the cancer cells expresses said cell surface antigen at a number of at least 64 cell surface antigen molecules per cell.

107. The immune cell of any one of claims 75 to 101 for the use of any one of claims 75 to 101, wherein a fraction of the cancer cells expresses said cell surface antigen at a number of at least 100 cell surface antigen molecules per cell.

108. The immune cell of any one of claims 75 to 101 for the use of any one of claims 75 to 101, wherein a fraction of the cancer cells expresses said cell surface antigen at a number of at least 200 cell surface antigen molecules per cell.

109. The immune cell of any one of claims 75 to 101 for the use of any one of claims 75 to 101, wherein a fraction of the cancer cells expresses said cell surface antigen at a number of at least 300 cell surface antigen molecules per cell.

110. The immune cell of any one of claims 75 to 109 for the use of any one of claims 75 to 109, wherein the cancer cells do not express said cell surface antigen at a number of more than 10,000 cell surface antigen molecules per cell.

111. The immune cell of any one of claims 75 to 109 for the use of any one of claims 75 to 109, wherein the cancer cells do not express said cell surface antigen at a number of more than 5,000 cell surface antigen molecules per cell.

112. The immune cell of any one of claims 75 to 109 for the use of any one of claims 75 to 109, wherein the cancer cells do not express said cell surface antigen at a number of more than 2,500 cell surface antigen molecules per cell.

113. The immune cell of any one of claims 75 to 109 for the use of any one of claims 75 to 109, wherein the cancer cells do not express said cell surface antigen at a number of more than 1,500 cell surface antigen molecules per cell.

114. The immune cell of any one of claims 75 to 109 for the use of any one of claims 75 to 109, wherein the cancer cells do not express said cell surface antigen at a number of more than 1,350 cell surface antigen molecules per cell.

115. The immune cell of any one of claims 75 to 109 for the use of any one of claims 75 to 109, wherein the cancer cells do not express said cell surface antigen at a number of more than 1,300 cell surface antigen molecules per cell.

116. The immune cell of any one of claims 75 to 109 for the use of any one of claims 75 to 109, wherein the cancer cells do not express said cell surface antigen at a number of more than 1,000 cell surface antigen molecules per cell.

117. The immune cell of any one of claims 75 to 109 for the use of any one of claims 75 to 109, wherein the cancer cells do not express said cell surface antigen at a number of more than 800 cell surface antigen molecules per cell.

118. The immune cell of any one of claims 75 to 109 for the use of any one of claims 75 to 109, wherein the cancer cells do not express said cell surface antigen at a number of more than 500 cell surface antigen molecules per cell.

119. The immune cell of any one of claims 75 to 118 for the use of any one of claims 75 to 118, wherein the treatment is a treatment in combination with myeloablative chemotherapy.

120. The immune cell of claim 119 for the use of claim 119, wherein the myeloablative chemotherapy comprises treatment with melphalan.

121. The immune cell of claim 120 for the use of claim 120, wherein melphalan at a dose between 100 mg per square meter and 200 mg per square meter, preferably wherein melphalan is to be administered at a dose of 140 mg per square meter.

122. The immune cell of any one of claims 75 to 121 for the use of any one of claims 75 to 121, wherein the treatment is a treatment in combination with autologous hematopoietic stem cell transplantation and/or wherein the treatment is a treatment in combination with allogeneic hematopoietic stem cell transplantation.

123. The immune cell of any one of claims 88 to 122 for the use of claims 88 to 122, wherein the chimeric antigen receptor is a chimeric antigen receptor having the amino acid sequence encoded by the nucleotide sequence of SEQ ID NO: 1 and/or SEQ ID NO: 3.

124. The immune cell of any one of claims 88 to 122 for the use of claims 88 to 122, wherein the chimeric antigen receptor is a chimeric antigen receptor having the amino acid sequence encoded by the nucleotide sequence of SEQ ID NO: 1.

125. The immune cell of any one of claims 88 to 122 for the use of claims 88 to 122, wherein the chimeric antigen receptor is a chimeric antigen receptor having the amino acid sequence encoded by the nucleotide sequence of SEQ ID NO: 3.
